(19) United States
(12) Patent Application Publication
(10) Pub. No.: US 2026/0125387 A1
(43) Pub. Date: May 7, 2026

(54) (R)-N-ETHYL-5-FLUORO-N-ISOPROPYL-2-((5-(2-(6-((2-METHOXYETHYL)(METHYL)AMINO)-2-METHYLHEXAN-3-YL)-2,6-DIAZASPIRO[3.4]OCTAN-6-YL)-1,2,4-TRIAZIN-6-YL)OXY)BENZAMIDE BESYLATE SALT

(71) Applicant: Janssen Pharmaceutica NV, Beerse (BE)

(72) Inventors: Wei CAI, Shanghai (CN); Xuedong DAI, Shanghai (CN); Olivier Alexis Georges QUEROLLE, Saint Vigor (FR); Johannes Wilhelmus J. THURING, Antwerpen (BE); Alicia Tee Fuay NG, Franklin Park, NJ (US); Nicolas Freddy Jacques BRUNO, Linkhout (BE); Robert Michael GEERTMAN, Beerse (BE); Dipali AHUJA, Beerse (BE); Yingtao LIU, Shanghai (CN); Vineet PANDE, Vosselaar (BE); Edward CLEATOR, Cambridgeshire (GB); Cyril BEN HAIM, Vosselaar (BE); Simon Jan C. SMOLDERS, Beerse (BE)

(21) Appl. No.: 18/570,727

(22) PCT Filed: Jun. 16, 2022
(86) PCT No.: PCT/CN2022/099089

§ 371 Date: Dec. 15, 2023

(30) Foreign Application Priority Data
Jun. 17, 2021 (WO) PCT/CN2021/100466
May 9, 2022 (WO) PCT/CN2022/091677

(51) Int. Cl. C07D 487/10; A61K 31/53; A61P 35/02; C07C 231/02

(52) U.S. Cl. CPC C07D 487/10; A61K 31/53; A61P 35/02; C07C 231/02

(57) ABSTRACT

The present invention relates to (R)—N-ethyl-5-fluoro-N-isopropyl-2-(5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt and solvates thereof.

This compound may be useful for therapy and/or prophylaxis in a mammal, pharmaceutical composition comprising such compound, and use as menin/MLL protein/protein interaction inhibitor, useful for treating diseases such as cancer, including but not limited to leukemia, myelodysplastic syndrome (MDS), and myeloproliferative neoplasms (MPN); and diabetes.

### SEQUENCE LISTING

The instant application contains a Sequence Listing which has been submitted electronically in ASCII format and is hereby incorporated by reference in its entirety. Said ASCII copy, created on Aug. 20, 2025 is named JAB7103USPCT3 SL.txt and is 6,392 bytes in size.

### FIELD OF THE INVENTION

The present invention relates to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt and solvates thereof.

This compound may be useful for therapy and/or prophylaxis in a mammal, pharmaceutical composition comprising such compound, and use as menin/MLL protein/protein interaction inhibitor, useful for treating diseases such as cancer, including but not limited to leukemia, myelodysplastic syndrome (MDS), and myeloproliferative neoplasms (MPN); and diabetes.

### BACKGROUND OF THE INVENTION

Chromosomal rearrangements affecting the mixed lineage leukemia gene (MLL; MLL1; KMT2A) result in aggressive acute leukemias across all age groups and still represent mostly incurable diseases emphasizing the urgent need for novel therapeutic approaches. Acute leukemias harboring these chromosomal translocations of MLL represent as lymphoid, myeloid or biphenotypic disease and constitute 5 to 10% of acute leukemias in adults and approximately 70% in infants (Marschalek, Br J Haematol 2011. 152(2), 141-54; Tomizawa et al., Pediatr Blood Cancer 2007. 49(2), 127-32).

MLL is a histone methyltransferase that methylates histone H3 on lysine 4 (H3K4) and functions in multiprotein complexes. Use of inducible loss-of-function alleles of Mill demonstrated that Mll1 plays an essential role in sustaining hematopoietic stem cells (HSCs) and developing B cells although its histone methyltransferase activity is dispensable for hematopoiesis (Mishra et al., Cell Rep 2014. 7(4), 1239-47).

Fusion of MLL with more than 60 different partners has been reported to date and has been associated with leukemia formation/progression (Meyer et al., Leukemia 2013. 27, 2165-2176). Interestingly, the SET (Su(var)3-9, enhancer of zeste, and trithorax) domain of MLL is not retained in chimeric proteins but is replaced by the fusion partner (Thiel et al., Bioessays 2012. 34, 771-80). Recruitment of chromatin modifying enzymes like Dot1L and/or the pTEFb complex by the fusion partner leads to enhanced transcription and transcriptional elongation of MLL target genes including HOXA genes (e.g. HOXA9) and the HOX cofactor MEIS1 as the most prominent ones. Aberrant expression of these genes in turn blocks hematopoietic differentiation and enhances proliferation.

Menin which is encoded by the Multiple Endocrine Neoplasia type 1 (MEN1) gene is expressed ubiquitously and is predominantly localized in the nucleus. It has been shown to interact with numerous proteins and is, therefore, involved in a variety of cellular processes. The best understood function of menin is its role as an oncogenic cofactor of MLL fusion proteins. Menin interacts with two motifs within the N-terminal fragment of MLL that is retained in all fusion proteins, MBM1 (menin-binding motif 1) and MBM2 (Thiel et al., Bioessays 2012. 34, 771-80). Menin/MLL interaction leads to the formation of a new interaction surface for lens epithelium-derived growth factor (LEDGF). Although MLL directly binds to LEDGF, menin is obligatory for the stable interaction between MLL and LEDGF and the gene specific chromatin recruitment of the MLL complex via the PWWP domain of LEDGF (Cermakova et al., Cancer Res 2014. 15, 5139-51; Yokoyama & Cleary, Cancer Cell 2008. 8, 36-46). Furthermore, numerous genetic studies have shown that menin is strictly required for oncogenic transformation by MLL fusion proteins suggesting the menin/MLL interaction as an attractive therapeutic target. For example, conditional deletion of Men1 prevents leukemogenesis in bone marrow progenitor cells ectopically expressing MLL fusions (Chen et al., Proc Natl Acad Sci 2006. 103, 1018-23). Similarly, genetic disruption of menin/MLL fusion interaction by loss-of-function mutations abrogates the oncogenic properties of the MLL fusion proteins, blocks the development of leukemia in vivo and releases the differentiation block of MLL-transformed leukemic blasts. These studies also showed that menin is required for the maintenance of HOX gene expression by MLL fusion proteins (Yokoyama et al., Cell 2005. 123, 207-18). In addition, small molecule inhibitors of menin/MLL interaction have been developed suggesting druggability of this protein/protein interaction and have also demonstrated efficacy in preclinical models of AML (Borkin et al., Cancer Cell 2015. 27, 589-602; Cierpicki and Grembecka, Future Med Chem 2014. 6, 447-462). Together with the observation that menin is not a requisite cofactor of MLL1 during normal hematopoiesis (Li et al., Blood 2013. 122, 2039-2046), these data validate the disruption of menin/MLL interaction as a promising new therapeutic approach for the treatment of MLL rearranged leukemia and other cancers with an active HOX/MEIS1 gene signature. For example, an internal partial tandem duplication (PTD) within the 5′ region of the MLL gene represents another major aberration that is found predominantly in de novo and secondary AML as well as myeloid dysplasia syndromes. Although the molecular mechanism and the biological function of MLL-PTD is not well understood, new therapeutic targeting strategies affecting the menin/MLL interaction might also prove effective in the treatment of MLL-PTD-related leukemias. Furthermore, castration-resistant prostate cancer has been shown to be dependent on the menin/MLL interaction (Malik et al., Nat Med 2015. 21, 344-52).

MLL protein is also known as Histone-lysine N-methyltransferase 2A (KMT2A) protein in the scientific field (UniProt Accession #Q03164).

Several references describe inhibitors targeting the menin-MLL interaction: WO2011029054, J Med Chem 2016, 59, 892-913 describe the preparation of thienopyrimidine and benzodiazepine derivatives; WO2014164543 describes thienopyrimidine and thienopyridine derivatives; Nature Chemical Biology March 2012, 8, 277-284 and Ren, J.; et al. Bioorg Med Chem Lett (2016), 26(18), 4472-4476 describe thienopyrimidine derivatives; J Med Chem 2014, 57, 1543-1556 describes hydroxy- and amninomethylpiperidine derivatives; Future Med Chem 2014, 6, 447-462 reviews small molecule and peptidomimetic compounds; WO2016195776 describes furo[2,3-d]pyrimidine, 911-purine, [1,3]oxazolo[5,4-d]pyrimidine, [1,3]oxazolo[4,5-d]pyrimidine, [1,3]thiazolo[5,4-d]pyrimidine, thieno[2,3-b]pyridine and thieno[2,3-d]pyrimidine derivatives; WO2016197027 describes 5,6,7,8-tetrahydropyrido[3,4-d]pyrimidine, 5,6,7,8-tetrahydropyrido]4,3-d]pyrimidine, pyrido[2,3-d]pyrimidine and quinoline derivatives; and WO2016040330 describes thienopyrimidine and thienopyridine compounds. WO2017192543 describes piperidines as Menin inhibitors. WO2017112768, WO2017207387, WO2017214367, WO2018053267 and WO2018024602 describe inhibitors of the menin-MLL interaction. WO2017161002 and WO2017161028 describe inhibitors of menin-MLL. WO2018050686, WO2018050684 and WO2018109088 describe inhibitors of the menin-MLL interaction. WO2018226976 describes methods and compositions for inhibiting the interaction of menin with MLL proteins. WO2018175746 provides methods of treatment for hematological malignancies and Ewing's sarcoma. WO2018106818 and WO2018106820 provide methods of promoting proliferation of a pancreatic cell. WO2018153312 discloses azaspiro compounds relating to the field of medicinal chemistry. WO2017132398 discloses methods comprising contacting a leukemia cell exhibiting an NPM1 mutation with a pharmacologic inhibitor of interaction between MLL and Menin. WO2019060365 describes substituted inhibitors of menin-MLL. WO2020069027 describes the treatment of hematological malignancies with inhibitors of menin. Krivtsov et al., Cancer Cell 2019. No. 6 Vol. 36, 660-673 describes a menin-MLL inhibitor.

### SUMMARY OF THE INVENTION

The present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt (benzenesulfonate salt):

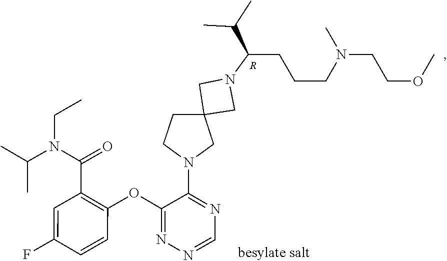

and solvates thereof.

A skilled person will understand that the ‘and solvates thereof’ refer to the besylate salt of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide. Thus the present invention covers the besylate salt of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide, and also the solvates of the besylate salt of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide.

In particular the present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt or hydrates thereof.

In particular the present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or solvates thereof.

In particular the present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or hydrates thereof.

In particular the present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 0.5-2.0 equivalents hydrate.

In particular the present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 2.0 equivalents hydrate.

More in particular the present invention is directed to a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate.

More in particular the present invention is directed to a crystalline form A of (R)—N-ethyl-5-fluoro-N-isopropyl-2-((5-(2-(6-((2-methoxyethyl)((methyl)amino)-2-methylhexan-3-yl)-2,6-diazaspiro[3.4]octan-6-yl)-1,2,4-triazin-6-yl)oxy)benzamide bis-besylate salt 0.5-2.0 equivalents hydrate.

More in particular the present invention is directed to a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt 2.0 equivalents hydrate.

The besylate salt of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide or a solvate thereof is superior with respect to its chemical/physical stability, its physical properties and the fact that it can be isolated as a stable crystalline solid.

An embodiment of the present invention is directed to a pharmaceutical composition comprising (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or solvates thereof.

The present invention also provides a pharmaceutical composition comprising, consisting of and/or consisting essentially of a pharmaceutically acceptable carrier, a pharmaceutically acceptable excipient, and/or a pharmaceutically acceptable diluent and (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or solvates thereof.

Also provided are processes for making a pharmaceutical composition comprising, consisting of, and/or consisting essentially of admixing (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or solvates thereof, and a pharmaceutically acceptable carrier, a pharmaceutically acceptable excipient, and/or a pharmaceutically acceptable diluent.

The present invention further provides methods for treating or ameliorating diseases such as cancer, including but not limited to leukemia, myelodysplastic syndrome (MDS), and myeloproliferative neoplasms (MPN); and diabetes, using (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or solvates thereof.

The present invention is also directed to the use of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt or solvates thereof in the preparation of a medicament wherein the medicament is prepared for treating a disease such as cancer, including but not limited to leukemia, myelodysplastic syndrome (MDS), and myeloproliferative neoplasms (MPN); and diabetes.

In particular, the compound according to the present invention and the pharmaceutical compositions thereof may be useful in the treatment or prevention of leukemias, in particular nucleophosmin (NPM1)-mutated leukemias, e.g. NPM1c.

In an embodiment, the compound according to the present invention, may have improved metabolic stability properties.

In an embodiment, the compound according to the present invention, may have extended in vivo half-life (T½).

In an embodiment, the compound according to the present invention, may have improved oral bioavailability.

In an embodiment, the compound according to the present invention, may reduce tumor growth e.g., tumours harbouring MLL (KMT2A) gene rearrangements/alterations and/or NPM1 mutations.

In an embodiment, the compound according to the present invention, may have improved PD properties in vivo during a prolonged period of time, e.g. inhibition of target gene expression such as MEIS1 and upregulation of differentiation marker over a period of at least 16 hours.

In an embodiment, the compound according to the present invention, may have an improved safety profile (e.g. reduced hERG inhibition; improved cardiovascular safety).

In an embodiment, the compound according to the present invention, may be suitable for Q.D. dosing (once daily).

The invention also relates to the use of the compound according to the present invention, in combination with an additional pharmaceutical agent for use in the treatment or prevention of cancer, including but not limited to leukemia, myelodysplastic syndrome (MDS), and myeloproliferative neoplasms (MPN); and diabetes.

Furthermore, the invention relates to a process for preparing a pharmaceutical composition according to the invention, characterized in that a pharmaceutically acceptable carrier is intimately mixed with a therapeutically effective amount of the compound according to the present invention.

The invention also relates to a product comprising the compound according to the present invention, and an additional pharmaceutical agent, as a combined preparation for simultaneous, separate or sequential use in the treatment or prevention of cancer, including but not limited to leukemia, myelodysplastic syndrome (MDS), and myeloproliferative neoplasms (MPN); and diabetes.

Additionally, the invention relates to a method of treating or preventing a cell proliferative disease in a warm-blooded animal which comprises administering to the said animal an effective amount of the compound according to the present invention, as defined herein, or a pharmaceutical composition or combination as defined herein.

In another embodiment, the present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt and solvates thereof for use as a medicament.

In another embodiment, the present invention is directed to (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt and solvates thereof for use as a medicament.

In another embodiment, the present invention is directed to a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt hydrate for use as a medicament.

In another embodiment, the present invention is directed to a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate for use as a medicament.

The present invention is also directed to the preparation of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt and solvates thereof.

The present invention is also directed to the preparation of a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate thereof.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific disclosure of the drawings. In the drawings:

FIG. 1 is an X-ray powder diffraction (XRPD) pattern of a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate.

FIG. 2: Efficacy study in Molm-14 subcutaneous (sc) model.

FIG. 3: Efficacy study in disseminated OCI-AML3 model.

FIG. 4 is an X-ray powder diffraction (XRPD) pattern of intermediate 234b.

### DETAILED DESCRIPTION OF THE INVENTION

The disclosure may be more fully appreciated by reference to the following description, including the following glossary of terms and the concluding examples. It is to be appreciated that certain features of the disclosed compound, crystalline form A, compositions and methods which are, for clarity, described herein in the context of separate aspects, may also be provided in combination in a single aspect. Conversely, various features of the disclosed compound, crystalline form A, compositions and methods that are, for brevity, described in the context of a single aspect, may also be provided separately or in any sub-combination.

Some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

Throughout the description and claims of this specification, the words “comprise” and “contain” and variations of the words, for example “comprising” and “comprises”, mean “including but not limited to”, and are not intended to (and do not) exclude other components.

For the purposes of this disclosure, the terms “crystalline form” and “polymorph” are synonymous. Characterizing information for crystalline forms is provided herein. It should be understood that the determination of a particular form can be achieved using any portion of the characterizing information that one skilled in the art would recognize as sufficient for establishing the presence of a particular form. For example, even a single distinguishing peak can be sufficient for one skilled in the art to appreciate that a particular form is present.

The term “isolated form” refers to a compound present in a form which is separate from any mixture with another compound(s), solvent system or biological environment. In an embodiment of the present invention, the crystalline form is present in an isolated form.

The term “room temperature” (RT) refers to a temperature of from about 15° C. to about 30° C., in particular from about 20° C. to about 30° C. Preferably, room temperature is a temperature of about 25° C.

When a crystalline form is identified using one or more XRPD peaks given as angles 20 (two theta), each of the 20 values is understood to mean the given value±0.2 degrees two theta, unless otherwise expressed.

The term “seeding” refers to the addition of crystalline material to a solution or mixture to initiate crystallisation or recrystallisation.

The term “compound of the (present) invention” or “compound according to the (present) invention” as used herein, is meant to include (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt and solvates thereof, or any subgroup thereof.

(R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt may exist as a solvate. A “solvate” may be a solvate with water (i.e., a hydrate) or with a common organic solvent.

(R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt or a solvate thereof may be provided in a substantially pure form, wherein the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt or a solvate thereof is present as a substantially pure form.

The crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate may be provided in a substantially pure form, wherein the mole percent of impurities in the isolated crystalline form is less than about 5 mole percent, preferably less than about 2 mole percent, more preferably, less than about 0.5 mole percent, most preferably, less than about 0.1 mole percent. In an embodiment of the present invention, (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate is present as a substantially pure form.

Also provided herein is a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate as a mixture with one or more additional forms of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide, including other crystalline forms, other salt forms, or solvates thereof. At least a particular weight percentage may be the crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate. Particular weight percentages include 10%, 20%, 30%, 40%, 50%, 60%, 70%, 80%, 85%, 90%, 95%, 96%, 97%, 98%, 99%, 99.5% and 99.9%.

Also provided herein is a process for preparing the crystalline form described herein, comprising the step of recrystallising Compound A, wherein the recrystallisation comprises the steps of:
a) adding Compound A, or a hydrate or solvate thereof, to a mixture of suitable solvents, in the presence of benzenesulfonic acid, and adjusting to a temperature in the range of from about 20° C. to solvent reflux temperature;
b) seeding with crystalline form A;
c) yielding a precipitate of the crystalline form described herein.

In particular, the mixture of suitable solvents in the process described in the previous paragraph is a mixture of acetone, water and IPAc.

In particular, the mixture of suitable solvents in the process described in the previous paragraph is a mixture of isopropanol, water and IPAc. In particular, the temperature used in the process is about 25° C.

Also provided is a crystalline form of

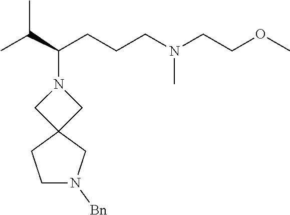 [SMILES: COCCN(C)CCC[C@H](C(C)C)N1CC2(CCN(Cc3ccccc3)C2)C1]

citric acid salt,
wherein the crystalline form produces an X-ray powder diffraction pattern comprising peaks at 5.82, 10.09 and 18.42 degrees two theta±0.2 degrees two theta; in particular wherein the X-ray powder diffraction pattern comprises peaks at 5.82, 8.52, 9.20, 10.09, 11.43, 13.61, 14.94, 15.89, 17.03 and 18.42 degrees two theta±0.2 degrees two theta.

Also provided is the following intermediate:

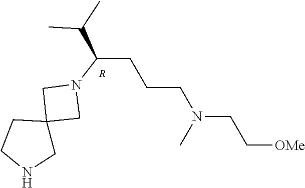

as a pharmaceutically acceptable salt, or a solvate thereof.

A skilled person will understand that the ‘or a solvate thereof’ refers to the pharmaceutically acceptable salt of the intermediate, thus covering a solvate of the pharmaceutically acceptable salt.

Also provided is the following intermediate:

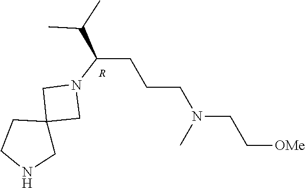

as a solvate.

Pharmaceutically acceptable salts include acid addition salts and base addition salts. Such salts may be formed by conventional means, for example by reaction of a free acid or a free base form with one or more equivalents of an appropriate base or acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic, tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

Appropriate base salt forms comprise, for example, the ammonium salts, the alkali and earth alkaline metal salts, e.g. the lithium, sodium, potassium, cesium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term solvate comprises the solvent addition forms. Examples of such solvent addition forms are e.g. hydrates, alcoholates and the like.

Also provided is a one step conversion from 5-fluoro-2-hydroxy-benzoic acid to N-ethyl-5-fluoro-2-hydroxy-N-isopropylbenzamide (intermediate 28):

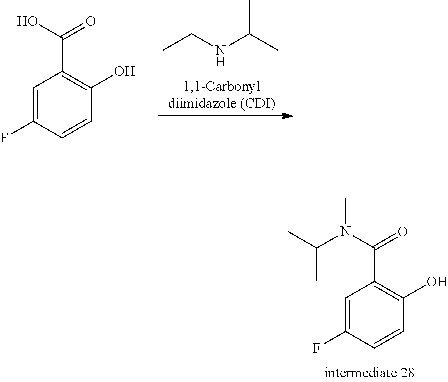

This reaction is performed in the presence of the coupling agent CDI, in a suitable solvent such as THF, toluene, acetonitrile or 2-methyltetrahydrofuran. In particular, the solvents is THF. The reaction is typically performed in a temperature range between 0° C. and reflux temperature, preferably between 0° C. and 50° C., more preferably between 10° C. and 30° C., even more preferably between 15° C. and 25° C.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

The term “subject” refers to an animal, preferably a mammal, most preferably a human, who has been the object of treatment, observation or experiment.

The term “therapeutically effective amount” as used herein, means that amount of active compound or pharmaceutical agent that elicits the biological or medicinal response in a tissue system, animal or human that is being sought by a researcher, veterinarian, medicinal doctor or other clinician, which includes alleviation or reversal of the symptoms of the disease or disorder being treated.

The term “composition” is intended to encompass a product comprising the specified ingredients in the specified amounts, as well as any product which results, directly or indirectly, from combinations of the specified ingredients in the specified amounts.

As used herein, unless otherwise noted, the term “affect” or “affected” (when referring to a disease, syndrome, condition or disorder that is affected by the inhibition of menin/MLL protein/protein interaction inhibitor) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

The terms “treatment” and “treating,” as used herein, are intended to refer to all processes wherein there may be a slowing, interrupting, arresting or stopping of the progression of a disorder, or amelioration of one or more symptoms thereof, but does not necessarily indicate a total elimination of all symptoms.

### Experimental Part

### Synthesis of the crystalline form A of (R)—N-ethyl-5-fluoro-N-isopropyl-2-((5-(2-(6-((2-methoxyethyl)(methyl)amino)-2-methylhexan-3-yl)-2,6-diazaspiro[13.4]octan-6-yl)-1,2,4-triazin-6-yl)oxy)benzamide bis-besylate salt hydrate

TABLE 1
Abbreviations

| Abbreviation | Meaning |
|---|---|
| Ag(Phen)₂OTf | silver triflate-bis(1,10-phenanthroline) complex |
| 2-MeTHF | 2-methyltetrahydrofuran |
| ACN or MeCN | acetonitrile |
| AcCl | acetyl chloride |
| AcOH | acetic acid |
| Ac₂O | acetic anhydride |
| aq. | aqueous |
| Ar | argon |
| BBr₃ | tribromoborane |
| bn | benzyl |
| Boc | tert-butyloxycarbonyl |
| Boc₂O | di-tert-butyl dicarbonate |
| n-BuLi | n-butyllithium |
| Cbz | benzyloxycarbonyl |
| CD₃OD | Methanol-d₄ |
| CHCl₃ | chloroform |
| Cs₂CO₃ | cesium carbonate |
| conc. | concentrated |
| DBU | 1,8-diazabicyclo[5.4.0]undec-7-ene |
| DCC | dicyclohexylcarbodiimide |
| DCE | dichloroethane |
| DCM | dichloromethane |
| DDQ | 4,5-dichloro-3,6-dioxocyclohexa-1,4-diene-1,2- |
| | dicarbonitrile |
| DEA | diethylamine |
| DIBAL-H | diisobutylaluminum hydride |
| DIEA or DIPEA | N,N-diisopropylethylamine |
| DMAP | N,N-dimethylpyridin-4-amine |
| DMF | N,N-dimethylformamide |
| DMP | Dess-Martin periodinane |
| DMSO | dimethyl sulfoxide |
| dppf | 1,1′-ferrocenediyl-bis(diphenylphosphine) |
| EDCI | N-(3-Dimethylaminopropyl)-N′-ethylcarbodiimide |
| | hydrochloride |
| EA or EtOAc | ethyl acetate |
| EtOH | ethanol |
| eq. | equivalent(s) |
| FA | formic acid |
| FCC | flash column chromatography |
| h | hour(s) |
| H₂ | hydrogen |
| HATU | 1-[bis(dimethylamino)methylene]-1H-1,2,3- |
| | triazolo[4,5-b]pyridinium 3-oxid hexafluoro- |
| | phosphate |
| H₂O | water |
| HCl | hydrochloric acid |
| HOBt | 1-Hydroxybenzotriazole |
| HPLC | high performance liquid chromatography |
| ICH₂Cl | chloroiodomethane |
| IPA | isopropyl alcohol |
| IPAc | isopropyl acetate |
| K₂CO₃ | potassium carbonate |
| KI | potassium iodide |
| K₂HPO₄ | dipotassium phosphate |
| K₃PO₄ | tripotassium phosphate |
| LiAlD₄ | lithium aluminum deuteride |
| LAH | lithium aluminum hydride |
| LiBH₄ | lithium borohydride |
| LDA | lithium diisopropylamide |
| LiCl | lithium chloride |
| LG | leaving group |
| Me | methyl |
| MeOH | methanol |
| 2-MeTHF | 2-methyltetrahydrofuran |
| min | minute(s) |
| mL | milliliters |
| mmol | millimoles |
| mg | milligram |
| MgSO₄ | magnesium sulfate |
| MSA | methanesulfonic acid |
| MsCl | methanesulfonyl chloride |
| MS | molecular sieve |
| MTBE | methyl tert-butyl ether |
| N₂ | nitrogen |
| NA | not available |
| NaBH₃CN | sodium cyanoborohydride |
| NaBH(OAc)₃ | sodium triacetoxyborohydride |
| NaBD₃CN | sodium cyanoborodeuteride |
| Na₂CO₃ | sodium carbonate |
| NaH | sodium hydride |
| NaHCO₃ | sodium bicarbonate |
| NaI | sodium iodide |
| NaOAc | sodium acetate |
| NaOH | sodium hydroxide |
| Na₂SO₃ | sodium sulfite |
| Na₂SO₄ | sodium sulfate |
| NH₄Cl | ammonium chloride |
| NMM | 1-4-Methylmorpholine |
| Pd₂dba₃ | tris(dibenzylideneacetone)dipalladium(0) |
| Pd(dppf)Cl₂•DCM | [1,1′-bis(diphenylphosphino)ferrocene] |
| | dichloropalladium(II), complex with dichlorome- |
| | thane |
| Pd(PPh₃)₄ | tetrakis(triphenylphosphine)palladium(0) |
| PE | petroleum ether |
| PG | protecting group |
| Phen | phenanthroline |
| psi | pound per square inch |
| p-TsOH | p-toluenesulfonic acid |
| p-TsOH•H₂O | p-toluenesulfonic acid monohydrate |
| Rₜ | retention time |
| Rochelle's salt | potassium sodium tartrate tetrahydrate |
| RT | room temperature |
| sat. | saturated |
| SFC | supercritical fluid chromatography |
| TBAF | tetrabutylammonium fluoride |
| TBDMS | tert-butyldimethylsilyl |
| TBDPS | tert-butyldiphenylsilyl |
| t-BuOK | potassium tert-butoxide |
| TEA | triethylamine |
| Tf | trifluoromethanesulfonyl |
| TFA | trifluoroacetic acid |
| THF | tetrahydrofuran |
| Ti(OiPr)₄ | titanium(IV) isopropoxide |
| TLC | thin layer chromatography |
| TMEDA | N,N,N′,N′-tetramethylethylenediamine |
| TMG | 1,1,3,3-tetramethylguanidine |
| TMSI | iodotrimethylsilane |
| Ts | p-toluenesulfonyl |
| TsCl | p-toluenesulfonyl chloride |
| v/v | volume per volume |
| vol. | volume(s) |
| wt | weight |
| Xantphos | 4,5-bis(diphenylphosphino)-9,9-dimethylxanthene |

A skilled person will realize that, even where not mentioned explicitly in the experimental protocols below, typically after a column chromatography purification, the desired fractions were collected and the solvent was evaporated.

In case no stereochemistry is indicated, this means it is a mixture of stereoisomers, unless otherwise is indicated or is clear from the context.

When a stereocenter is indicated with ‘RS’ this means that a racemic mixture was obtained at the indicated centre, unless otherwise indicated.

As understood by a person skilled in the art, compounds and intermediates synthesized using the protocols as indicated may exist as a solvate e.g. hydrate, and/or contain residual solvent or minor impurities. Compounds or intermediates isolated as a salt form or solvates (e.g. hydrates), may be integer stoichiometric i.e. mono- or di-salts, or of intermediate stoichiometry. When an intermediate or compound is indicated as ‘HCl salt’ without indication of the number of equivalents of HCl, this means that the number of equivalents of HCl was not determined. When an intermediate or compound is indicated as ‘hydrate’ without indication of the number of equivalents of H₂O, this means that the number of equivalents of H₂O was not determined.

For convenience (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (free base) is indicated as “Compound A” in the experimental part below.

### Example 1—Synthesis of (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A)—preparation method A

### Preparation of Intermediate 1

### tert-butyl (5-methyl-4-oxohexyl)carbamate

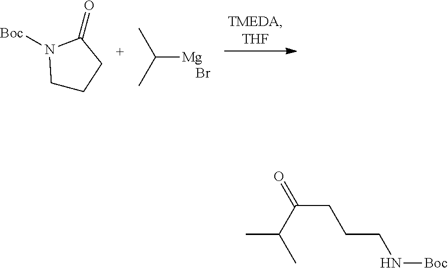

To a solution of tert-butyl 2-oxopyrrolidine-1-carboxylate (5.0 g, 27 mmol) and TMEDA (5.0 mL, 33 mmol) in THF (60 mL) cooled at −70° C. was slowly added isopropylmagnesium bromide solution (19 mL, 55 mmol, 2.9 M in 2-methyltetrahydrofuran), the resulting mixture was slowly warmed to RT and stirred for 12 h. The mixture was poured into sat. aq. NH₄Cl (50 mL) solution and extracted with EtOAc (50 mL×3). The combined organic layers were dried over anhydrous Na₂SO₄, filtered, and concentrated under reduced pressure to give the crude product, which was further purified by FCC (PE/EtOAc=1:0 to 100:1) to afford the title intermediate (3.7 g, 60% yield) as a yellow oil.

### Preparation of Intermediate 13

### tert-butyl 6-(3,6-dichloro-1,2,4-triazin-5-yl)-2,6-diazaspiro[3.4]octane-2-carboxylate

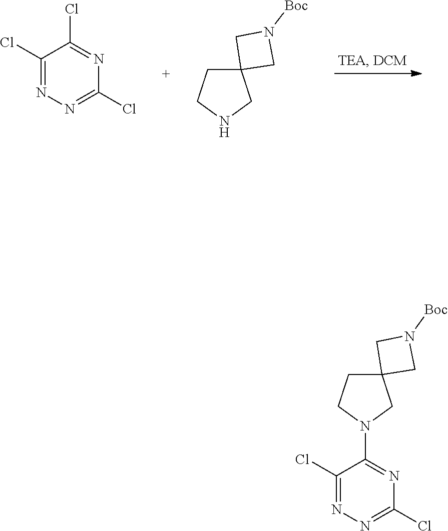 [SMILES: Clc1nnc(Cl)c(Cl)n1 ; CC(C)(C)OC(=O)N1CC2(CCNC2)C1 ; CC(C)(C)OC(=O)N1CC2(CCN(c3nc(Cl)nnc3Cl)C2)C1]

To the solution of 3,5,6-trichloro-1,2,4-triazine (10.0 g, 54.2 mmol) and TEA (15.2 mL, 109 mmol) in DCM (100 mL) cooled at 0° C. was added tert-butyl 2,6-diazaspiro[3.4]octane-2-carboxylate (9.21 g, 43.4 mmol), the mixture was warmed to RT and stirred for 1 h. The mixture was diluted with water (20 mL) and extracted with DCM (30 mL×3). The combined organic layers were washed with brine, dried over Na₂SO₄, filtered and concentrated under reduced pressure to give the crude product which was purified by FCC on silica gel (PE/EtOAc=1:0 to 3:1) to afford the title intermediate (12.0 g, 58% yield) as a yellow solid.

### Preparation of Intermediate 27

### N-ethyl-5-fluoro-N-isopropyl-2-methoxybenzamide

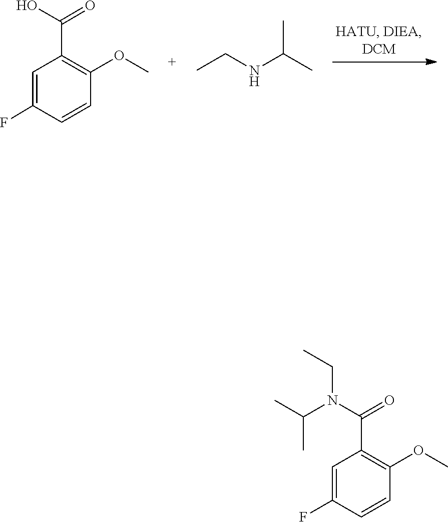

To the mixture of 5-fluoro-2-methoxybenzoic acid (8.00 g, 47.0 mmol) and N-ethylpropan-2-amine (8.19 g, 94.0 mmol) in dry DCM (150 mL) cooled at 0° C., were slowly added HATU (21.5 g, 56.5 mmol) and DIEA (9.10 g, 70.4 mmol) in portions. The resulting mixture was slowly warmed to RT and stirred for 8 h. The organic layer was washed with water (20 mL×3) and dried over anhydrous Na₂SO₄. After filtration, the solvent was removed under reduced pressure and the crude product was purified by FCC (EtOAc/PE=0% to 20%) to afford the title intermediate (12.0 g, 96% yield) as a white solid.

### Preparation of Intermediate 28

### N-ethyl-5-fluoro-2-hydroxy-N-isopropylbenzamide

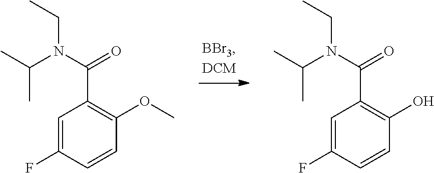 [SMILES: CCN(C(=O)c1cc(F)ccc1OC)C(C)C ; CCN(C(=O)c1cc(F)ccc1O)C(C)C]

To the solution of N-ethyl-5-fluoro-N-isopropyl-2-methoxybenzamide (intermediate 27) (12.0 g, 50.1 mmol) in dry DCM (100 mL) cooled at −78° C. was slowly added BBr₃ (14.4 mL, 152 mmol), the resulting mixture was slowly warmed to RT and stirred for 8 h. The mixture was cooled to −78° C. again and MeOH (5 mL) was added dropwise to quench the reaction. The resulting mixture was slowly warmed to RT and the pH value was adjusted to about 8 by adding sat. aq. NaHCO₃ solution. The aqueous layer was extracted by DCM (50 mL×3) and the combined organic layers were dried over anhydrous Na₂SO₄, filtered and concentrated under reduced pressure to give the crude product which was purified by FCC (EtOAc/PE=0% to 20%) to afford the title intermediate (9.0 g, 78% yield) as a white solid.

### Alternative Preparation of Intermediate 28

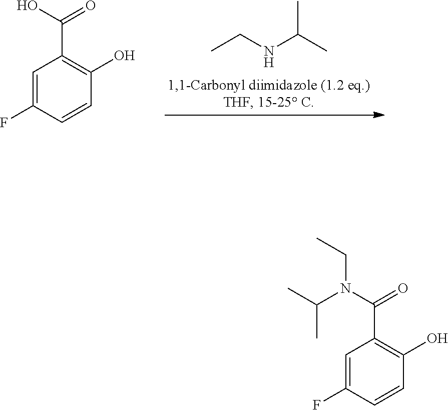

A mixture of 5-fluoro-2-hydroxy-benzoic acid (14.0 kg, 89.68 mol, 1.0 equiv.) in THF (168 L, 12 volumes) was adjusted to between 15-25° C., and 1,1-carbonyldiimidazole, (17.45 kg, 107.62 mol, 1.2 equiv.) was added over a period of 1 hour. After addition, the mixture was stirred for 18 hours at 15-25° C. After this time N-ethylpropan-2-amine (14.85 kg, 170.39 mol, 1.9 equiv.) was added to the mixture at 15-25° C. over a period of 2 hours. The resulting mixture was further aged for between 18-24 hours at 15-25° C. The pH was the adjusted to between pH4-5 with aq. 10% H₂SO₄ (140 kg, 10 volumes) and the layers were separated. The organic phase was concentrated to between 42-56 L maintaining a temperature below 40° C., and then n-heptane (43 kg, 4.5 volumes) was added to the mixture at 15-25° C. over a period of 3 hours. The mixture was then cooled to 0-10° C. and stirred for an additional 6 hours. The resulting slurry was filtered and the cake was washed with a tert-butyl methyl ether (MTBE):n-heptane mixture (25 kg of a 2:3 volume/volume mixture of MTBE:n-heptane, 2.5 volumes). The cake wash was repeated a further two times and the resulting solid was dried in-vacuo at 50° C. to afford intermediate 28 (16.5 kg, purity: 99.1%,yield: 80.4%).

### Preparation of Intermediate 14

### tert-butyl 6-(3-chloro-6-(2-(ethyl(isopropyl)carbamoyl)-4-fluorophenoxy)-1,2,4-triazin-5-yl)-2,6-diazaspiro[3.4]octane-2-carboxylate

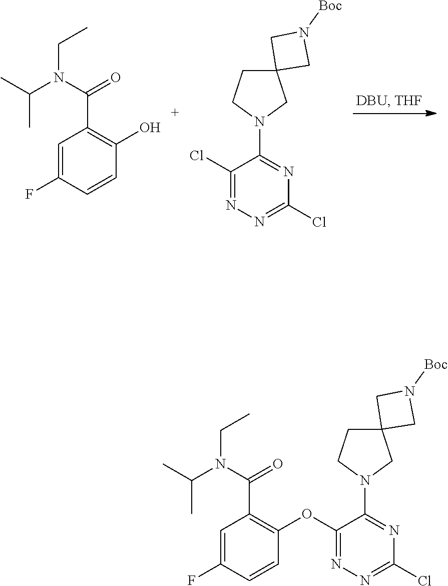 [SMILES: CCN(C(=O)c1cc(F)ccc1O)C(C)C ; CC(C)(C)OC(=O)N1CC2(CCN(c3nc(Cl)nnc3Cl)C2)C1 ; CCN(C(=O)c1cc(F)ccc1Oc1nnc(Cl)nc1N1CCC2(CN(C(=O)OC(C)(C)C)C2)C1)C(C)C]

The mixture of tert-butyl 6-(3,6-dichloro-1,2,4-triazin-5-yl)-2,6-diazaspiro[3.4]octane-2-carboxylate (intermediate 13) (12.0 g, 33.3 mmol), N-ethyl-5-fluoro-2-hydroxy-N-isopropylbenzamide (intermediate 28) (7.5 g, 33.3 mmol) and DBU (6.1 g, 40.1 mmol) in THF (120 mL) was stirred at 25° C. for 8 h. The mixture was diluted with water (30 mL) and extracted with DCM (30 mL×3). The combined organic layers were washed with brine, dried over Na₂SO₄, filtered and concentrated under reduced pressure to give the crude product which was purified by FCC (PE/EtOAc=1:0 to 3:1) to afford the title intermediate (14.0 g, 73% yield) as green solid.

### Preparation of Intermediate 2

### tert-butyl 6-(6-(2-(ethyl(isopropyl)carbamoyl)-4-fluorophenoxy)-1,2,4-triazin-5-yl)-2,6-diazaspiro[3.4]octane-2-carboxylate

### Synthesis Method A for Intermediate 2

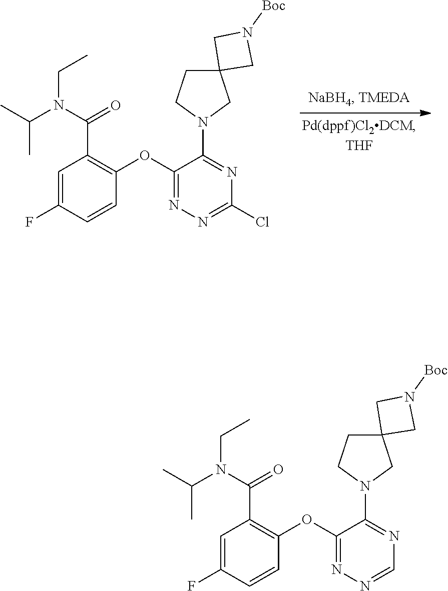 [SMILES: CCN(C(=O)c1cc(F)ccc1Oc1nnc(Cl)nc1N1CCC2(CN(C(=O)OC(C)(C)C)C2)C1)C(C)C ; CCN(C(=O)c1cc(F)ccc1Oc1nncnc1N1CCC2(CN(C(=O)OC(C)(C)C)C2)C1)C(C)C]

To the mixture of tert-butyl 6-(3-chloro-6-(2-(ethyl(isopropyl)carbamoyl)-4-fluorophenoxy)-1,2,4-triazin-5-yl)-2,6-diazaspiro[3.4]octane-2-carboxylate (intermediate 14) (20 g, 36.4 mmol), NaBH₄ (2.48 g, 65.7 mmol) and TMEDA (8.54 g, 73.5 mmol) in THF (500 mL) was added Pd(dppf)Cl₂·DCM (1.70 g, 2.08 mmol) under N₂ atmosphere. After addition, the reaction mixture was stirred at 25° C. for 14 h. The reaction mixture was filtered and the filtrate was concentrated, the residue was purified by FCC on silica gel (EtOAc) to afford the title intermediate (15 g, 93% purity, 74% yield) as brown solid.

### Synthesis Method B for Intermediate 2

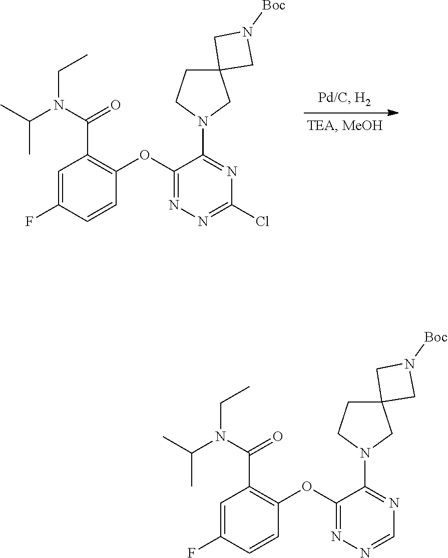 [SMILES: CCN(C(=O)c1cc(F)ccc1Oc1nnc(Cl)nc1N1CCC2(CN(C(=O)OC(C)(C)C)C2)C1)C(C)C ; CCN(C(=O)c1cc(F)ccc1Oc1nncnc1N1CCC2(CN(C(=O)OC(C)(C)C)C2)C1)C(C)C]

To the solution of tert-butyl 6-(3-chloro-6-(2-(ethyl(isopropyl)carbamoyl)-4-fluorophenoxy)-1,2,4-triazin-5-yl)-2,6-diazaspiro[3.4]octane-2-carboxylate (intermediate 14) (22.0 g, 40.1 mmol), TEA (15 mL) in MeOH (100 mL) was added Pd/C (wet, 5.0 g, 10%) The resulting mixture was stirred under H₂ atmosphere (30 psi) at 25° C. for 8 h. The reaction mixture was filtered through a celite pad and the filtrate was concentrated in vacuo to afford the title intermediate (25.0 g, crude), which was used directly in next step without further purification.

### Preparation of Intermediate 3

### 2-((5-(2,6-diazaspiro[3.4]octan-6-yl)-1,2,4-triazin-6-yl)oxy)-N-ethyl-5-fluoro-N-isopropylbenzamide

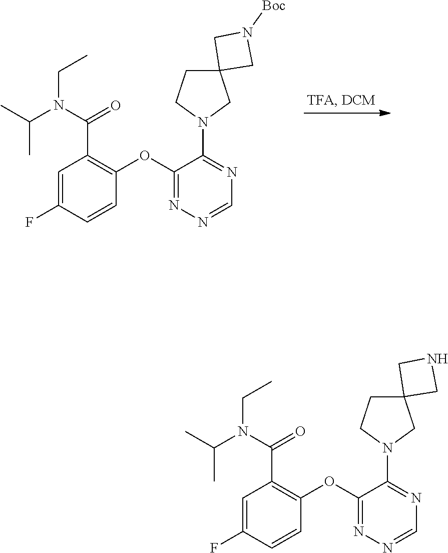 [SMILES: CCN(C(=O)c1cc(F)ccc1Oc1nncnc1N1CCC2(CN(C(=O)OC(C)(C)C)C2)C1)C(C)C ; CCN(C(=O)c1cc(F)ccc1Oc1nncnc1N1CCC2(CNC2)C1)C(C)C]

To the solution of tert-butyl 6-(6-(2-(ethyl(isopropyl)carbamoyl)-4-fluorophenoxy)-1,2,4-triazin-5-yl)-2,6-diazaspiro[3.4]octane-2-carboxylate (intermediate 2) (300 mg, 0.583 mmol) in DCM (5 mL) was added TFA (0.5 mL, 6.4 mmol), the resulting mixture was stirred at RT for 3 h. Then 10% NaOH (5 mL) solution was slowly added into the mixture to adjust the pH value to about 12, the resulting mixture was extracted with DCM (10 mL×3). The combined organic layers were dried over anhydrous Na₂SO₄, filtered, and concentrated in vacuo to afford the title intermediate (220 mg, 90% yield) as a white solid.

### Preparation of Compound 61

### tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate

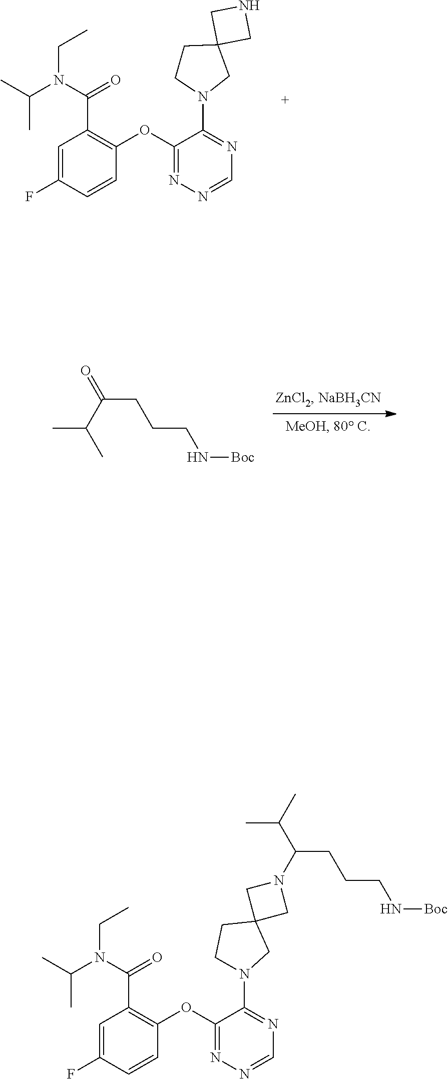 [SMILES: CCN(C(=O)c1cc(F)ccc1Oc1nncnc1N1CCC2(CNC2)C1)C(C)C ; CC(C)C(=O)CCCNC(=O)OC(C)(C)C ; CCN(C(=O)c1cc(F)ccc1Oc1nncnc1N1CCC2(C1)CN(C(CCCNC(=O)OC(C)(C)C)C(C)C)C2)C(C)C]

The mixture 2-((5-(2,6-diazaspiro[3.4]octan-6-yl)-1,2,4-triazin-6-yl)oxy)-N-ethyl-5-fluoro-N-isopropylbenzamide (intermediate 3) (1.0 g, 2.4 mmol), tert-butyl (5-methyl-4-oxohexyl)carbamate (intermediate 1) (830 mg, 3.62 mmol) and ZnCl₂ (660 mg, 4.84 mmol) in MeOH (15 mL) was stirred at 80° C. for 0.5 h. Then NaBH₃CN (310 mg, 4.93 mmol) was added and the resulting mixture was stirred at 80° C. for 6 h. After cooled to RT, the mixture was concentrated under reduced pressure to give the crude product, which was further purified by preparative HPLC using a Waters Xbridge Prep OBD (column: C18 150×40 mm 10 um; eluent: ACN/H₂O (0.05% ammonia) from 45% to 75% v/v) to afford the title compound (700 mg, 46% yield) as colorless oil.

### Preparation of Compounds 62 and 63

### tert-butyl (R)-(4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate

### tert-butyl (S)-(4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate

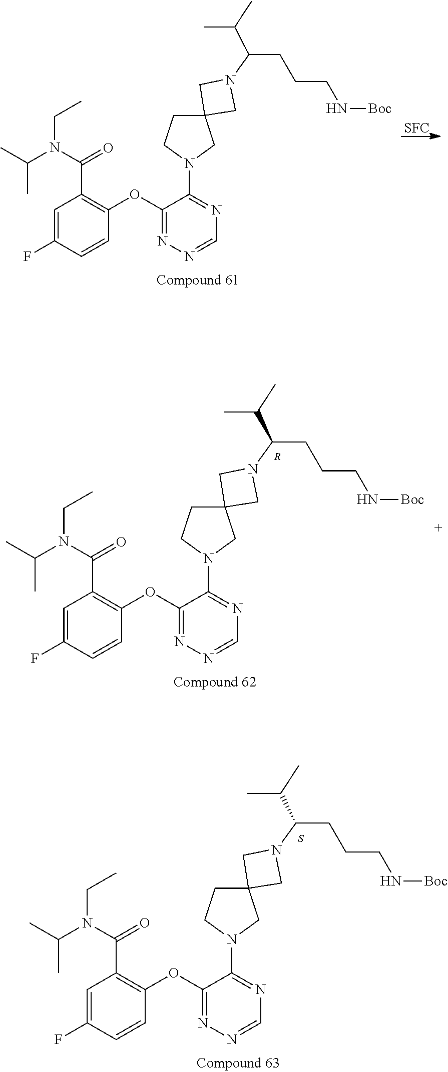

tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate (Compound 61) (200 mg, 0.319 mmol) was purified by SFC over DAICEL CHIRALPAK IG (column: 250×30 mm 10 um; isocratic elution: EtOH (containing 0.1% of 25% ammonia): supercritical CO₂, 40%:60% (v/v)) to afford the title compounds (Compound 62) (85 mg, 42% yield) and (Compound 63) (80 mg, 40% yield) both as light yellow oil.

### Compound 64

### (R)-2-((5-(2-(6-amino-2-methylhexan-3-yl)-2,6-diazaspiro[3.4]octan-6-yl)-1,2,4-triazin-6-yl)oxy)-N-ethyl-5-fluoro-N-isopropylbenzamide

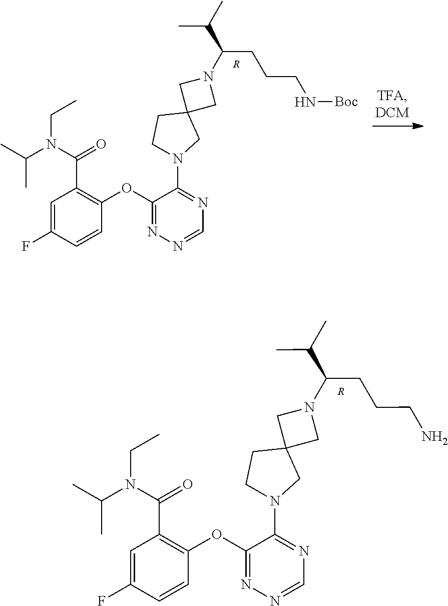

To the solution of tert-butyl (R)-(4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)carbamate (Compound 62) (550 mg, 0.876 mmol) in DCM (4 mL) was slowly added TFA (4 mL), and the resulting mixture was stirred at 25° C. for 1 h. The reaction mixture was concentrated under reduced pressure to give a residue. The residue was diluted in DCM (40 mL) and the pH value was adjusted to around 12 by aq. NaOH (2 M, 16 mL) solution. The aqueous layer was extracted with DCM (10 mL×2). The combined organic layers were dried over anhydrous Na₂SO₄, filtered and concentrated in vacuo to afford the title compound (460 mg, crude) as yellow solid, which was used directly in next step without further purification.

### Compound 11

### (R)—N-ethyl-5-fluoro-N-isopropyl-2-((5-(2-(6-((2-methoxyethyl)amino)-2-methylhexan-3-yl)-2,6-diazaspiro[3.4]octan-6-yl)-1,2,4-triazin-6-yl)oxy)benzamide

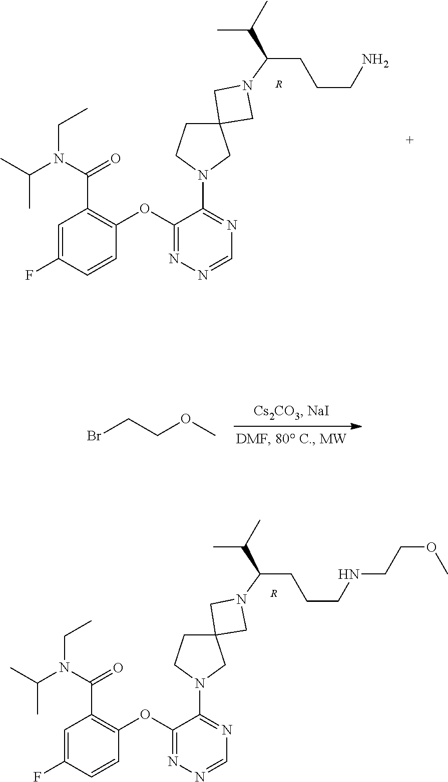

The mixture of (R)-2-((5-(2-(6-amino-2-methylhexan-3-yl)-2,6-diazaspiro[3.4]octan-6-yl)-1,2,4-triazin-6-yl)oxy)-N-ethyl-5-fluoro-N-isopropylbenzamide (Compound 64) (120 mg, crude), 1-bromo-2-methoxyethane (32 mg, 0.23 mmol), Cs₂CO₃ (222 mg, 0.681 mmol), NaI (102 mg, 0.680 mmol) in DMF (1 mL) was stirred at 80° C. via microwave irradiation for 1 h. After cooling to RT, the mixture was diluted with H₂O (10 mL) and extracted with EtOAc (3×10 mL). The combined organic layers were washed with H₂O (10 mL), dried over Na₂SO₄, filtered and concentrated under reduced pressure to afford the crude product which was further purified by HPLC over a Phenomenex Gemini-NX (column: 150×30 mm 5 μm; eluent: ACN/H₂O (10 mM NH₄HCO₃) from 51% to 71% (v/v)) and further purified by SFC over DAICEL CHIRALCEL OD-H (column: 250×30 mm 5 um; eluent: supercritical CO₂ in EtOH (0.1% v/v ammonia) 25/25, v/v) to afford the title compound (5.13 mg, 96% purity) as yellow solid.

LC-MS (ESI) (Method 1): Rₜ=2.997 min, m/z found 586.3 [M+H]⁺.

### Compound A

### (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide

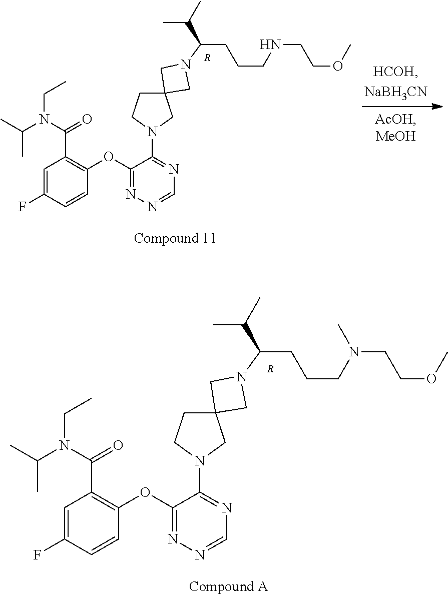

The mixture of (R)—N-ethyl-5-fluoro-N-isopropyl-2-((5-(2-(6-((2-methoxyethyl)amino)-2-methylhexan-3-yl)-2,6-diazaspiro[3.4]octan-6-yl)-1,2,4-triazin-6-yl)oxy)benzamide (Compound 11) (40.0 mg, 0.068 mmol), formaldehyde (55.4 mg, 0.683 mol, 37% in water) and AcOH (8.2 mg, 0.137 mmol) in anhydrous MeOH (2 mL) was stirred at 45° C. for 1 h. Then, NaBH₃CN (8.6 mg, 0.137 mmol) was added to the mixture and the resulting mixture was stirred at 45° C. for another 1 h. After cooling to RT, the reaction mixture was treated with sat. aq. NaHCO₃ (40 mL) to adjust the pH value to about 8 and further extracted with DCM (20 mL×3). The combined organic layers were dried over anhydrous Na₂SO₄, filtered and concentrated under reduced pressure to give the crude which was purified by preparative HPLC over Boston Prime (column: C18 150×30 mm Sum, Mobile Phase A: H₂O (0.04% ammonia+10 mM NH₄HCO₃), Mobile Phase B: ACN, Flow rate: 25 mL/min, gradient condition B/A from 50% to 80% (50% B to 80% B)) to afford the title compound (9.62 mg, 99.10% purity, 23.3% yield) as yellow oil.

### Example 2—Synthesis of (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A)—preparation method B

### Preparation of Intermediate 7

### 4-((tert-butoxycarbonyl)(methyl)amino)butanoic acid

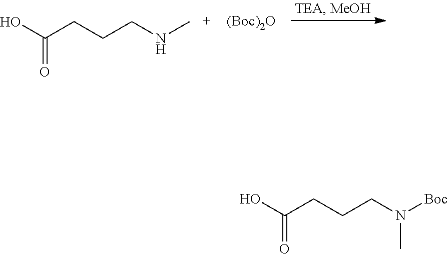

To a solution of 4-(methylamino)butanoic acid hydrochloride (3.0 g, 19.5 mmol) and TEA (7.78 mL, 58.6 mmol) in MeOH (30 mL) was added Boc₂O (4.69 g, 21.5 mmol) dropwise. The mixture was stirred at RT for 2 h. The mixture was concentrated under reduced pressure and the residue was diluted with EtOAc (100 mL), washed with cooled 0.1 N HCl (70 mL×2), H₂O (50 mL×2) and brine (50 mL), dried over Na₂SO₄, filtered and concentrated to afford the title intermediate (1.80 g, crude) as colorless oil.

### Preparation of Intermediate 8

### tert-butyl (4-(methoxy(methyl)amino)-4-oxobutyl)(methyl)carbamate

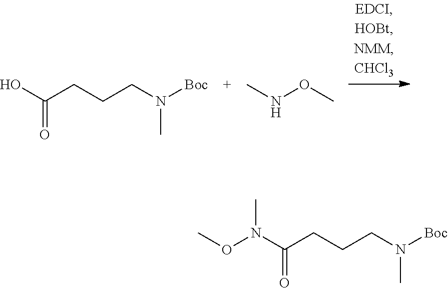

To a solution of 4-((tert-butoxycarbonyl)(methyl)amino)butanoic acid (intermediate 7) (1.80 g, crude) in CHCl₃ (30 mL) was added N,O-dimethylhydroxylamine hydrochloride (960 mg, 9.84 mmol), HOBt (1.24 g, 9.18 mmol) and NMM (2.80 mL, 25.1 mmol). And, then EDCI (2.23 g, 11.6 mmol) was added and the reaction mixture was stirred at RT for 4 h. The reaction mixture was diluted with DCM (100 mL), washed with 1N HCl (30 mL×3), sat. aq. NaHCO₃ (30 mL×3) and brine (30 mL), dried over Na₂SO₄, filtered and concentrated under in vacuo to afford the title intermediate (1.70 g, crude) as colorless oil.

### Preparation of Intermediate 9

### tert-butyl methyl(5-methyl-4-oxohexyl)carbamate

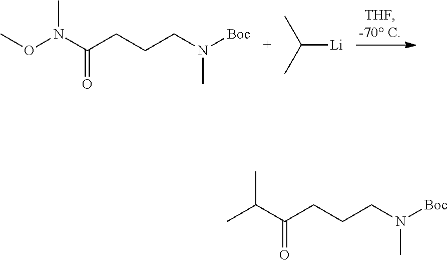

To a solution of tert-butyl (4-(methoxy(methyl)amino)-4-oxobutyl)(methyl)carbamate (intermediate 8) (200 mg, crude) in THF (5 mL) cooled at −70° C. under N₂ atmosphere was added dropwise isopropyllithium (3.2 mL, 2.24 mmol, 0.7M in pentane). The resulting mixture was stirred at −70° C. for 2 h. The mixture was quenched with sat. aq. NH₄Cl (15 mL), extracted with EtOAc (30 mL×2). The combined organic layers were washed with brine (30 mL), dried over Na₂SO₄, filtered and concentrated under reduced pressure to give a crude product. The crude product was further purified by FCC (PE/EtOAc=10:1) to afford the title intermediate (60 mg) as colorless oil.

### Preparation of Compound 60

### tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)(methyl)carbamate

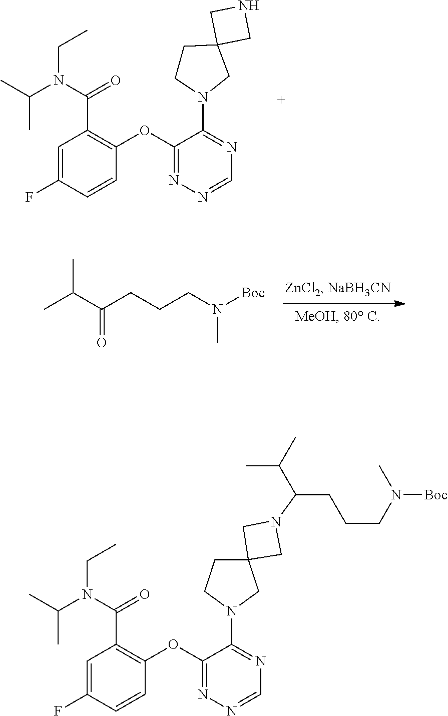 [SMILES: CCN(C(=O)c1cc(F)ccc1Oc1nncnc1N1CCC2(CNC2)C1)C(C)C ; CC(C)C(=O)CCCN(C)C(=O)OC(C)(C)C ; CCN(C(=O)c1cc(F)ccc1Oc1nncnc1N1CCC2(C1)CN(C(CCCN(C)C(=O)OC(C)(C)C)C(C)C)C2)C(C)C]

To a solution of 2-((5-(2,6-diazaspiro[3.4]octan-6-yl)-1,2,4-triazin-6-yl)oxy)-N-ethyl-5-fluoro-N-isopropylbenzamide (intermediate 3) (600 mg, 1.45 mmol) and tert-butyl methyl(5-methyl-4-oxohexyl)carbamate (intermediate 9) (330 mg, 1.37 mmol) in MeOH (50 mL) was added ZnCl₂ (789 mg, 5.79 mmol). The resulting mixture was stirred at 80° C. for 2 h. Then NaBH₃CN (729 mg, 11.6 mmol) was added and the reaction mixture was stirred at 80° C. overnight. After cooling to RT, the mixture was concentrated under reduced pressure to give a crude residue, which was diluted with DCM (50 mL), quenched with sat. aq. NH₄Cl (50 mL) and extracted with DCM (50 mL×3). The combined organic layers were washed with brine (50 mL), dried over Na₂SO₄, filtered and the filtrate was concentrated under reduced pressure to give a crude product which was further purified by FCC (DCM/MeOH=10:1) to afford the title compound (400 mg, 42% yield) as white solid.

### Compound 67

### N-ethyl-5-fluoro-N-isopropyl-2-((5-(2-(2-methyl-6-(methylamino)hexan-3-yl)-2,6-diazaspiro[3.4]octan-6-yl)-1,2,4-triazin-6-yl)oxy)benzamide hydrochloride

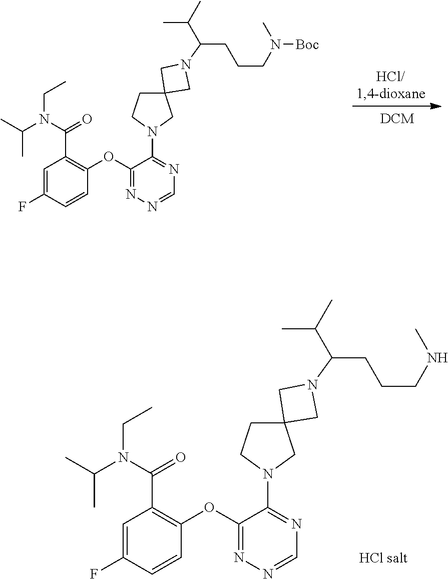 [SMILES: CCN(C(=O)c1cc(F)ccc1Oc1nncnc1N1CCC2(C1)CN(C(CCCN(C)C(=O)OC(C)(C)C)C(C)C)C2)C(C)C ; CCN(C(=O)c1cc(F)ccc1Oc1nncnc1N1CCC2(C1)CN(C(CCCNC)C(C)C)C2)C(C)C]

To a solution of tert-butyl (4-(6-(6-(2-(ethyl(isopropyl)carbamoyl)-4-fluorophenoxy)-1,2,4-triazin-5-yl)-2,6-diazaspiro[3.4]octan-2-yl)-5-methylhexyl)(methyl)carbamate (Compound 60) (1 g, 1.56 mmol) in DCM (10 mL) was added 4M HCl in dioxane (5 mL, 20 mmol), the resulting mixture was stirred at RT for 1 h. The reaction mixture was concentrated in vacuo to afford the title compound (960 mg, crude, HCl salt) which was used directly in next step without further purification.

### Compound A

### (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide

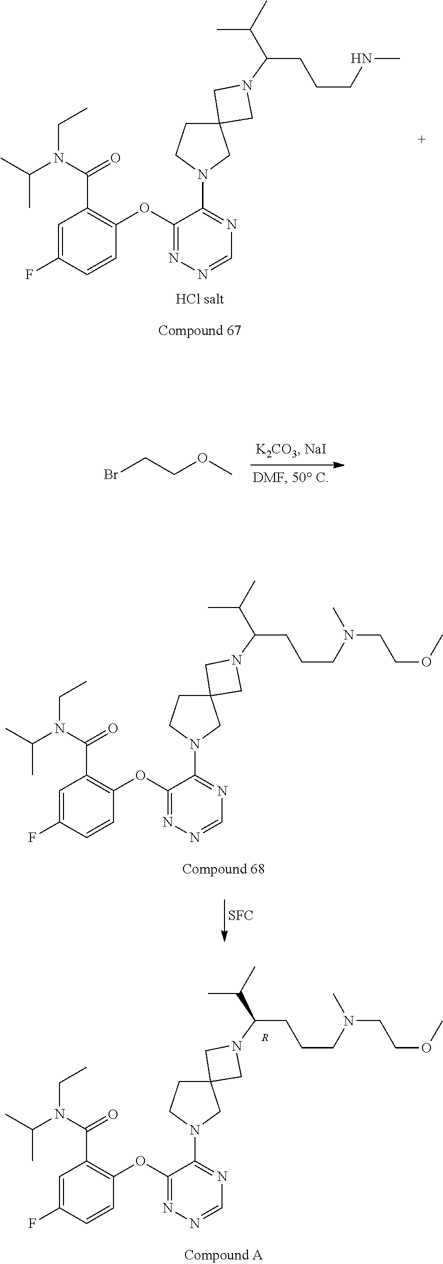

To the mixture of N-ethyl-5-fluoro-N-isopropyl-2-((5-(2-(2-methyl-6-(methylamino) hexan-3-yl)-2,6-diazaspiro[3.4]octan-6-yl)-1,2,4-triazin-6-yl)oxy)benzamide hydrochloride (Compound 67) (480 mg, crude), K₂CO₃ (700 mg, 5.07 mmol) and NaI (400 mg, 2.67 mmol) in DMF (5 mL) was added 1-bromo-2-methoxyethane (230 mg, 1.65 mmol). The resulting mixture was stirred at 50° C. overnight. After cooled to RT, the reaction mixture was quenched with H₂O (30 mL) and extracted with DCM (30 mL×3). The combined organic layers were washed with brine (30 mL×3), dried over Na₂SO₄, filtered and concentrated to give a crude residue. The residue was purified by FCC (DCM/MeOH=10:1) to afford N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide (Compound 68) (250 mg, 48% yield) as yellow oil.

The N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide (Compound 68) (960 mg, combined from several batches obtained by Method B) was first separated by SFC using DAICEL CHIRALPAK IG (column: 250×30 mm 10 um; Mobile phase: A: Supercritical CO₂, B: EtOH (0.1% ammonia), A:B=40:60 at 60 mL/min) and further purified by preparative HPLC using Boston Prime (column: 150×30 mm Sum, Mobile Phase A: H₂O (10 mM NH₄HCO₃), Mobile Phase B: ACN, Flow rate: 25 mL/min, gradient condition B/A from 55% to 85%) to afford the title compound (270 mg) as colorless oil.

¹H NMR (400 MHz, Methanol-d₄): δ=8.40 (s, 1H), 7.47-7.32 (m, 1H), 7.30-7.10 (m, 2H), 4.24-4.01 (m, 2H), 3.89-3.60 (m, 3H), 3.48 (br s, 3H), 2.63-2.51 (m, 2H), 2.43-2.32 (m, 2H), 2.29-2.07 (m, 6H), 1.86-1.72 (m, 1H), 1.62-1.44 (m, 2H), 1.39-1.02 (m, 10H), 0.99-0.66 (m, 9H). Some protons were hidden by the solvent peak and are not reported.

LCMS (ESI) (Method 2): Rₜ=1.965 min, m/z found 600.3 [M+H]⁺.

SFC (Method 11): Rₜ=4.904 min.

### Example 3—Synthesis of (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A)—preparation method C

### Preparation of Intermediate 227

### tert-butyl (R)-(1-(2,2-dimethyl-4,6-dioxo-1,3-dioxan-5-yl)-3-methylbutan-2-yl)carbamate

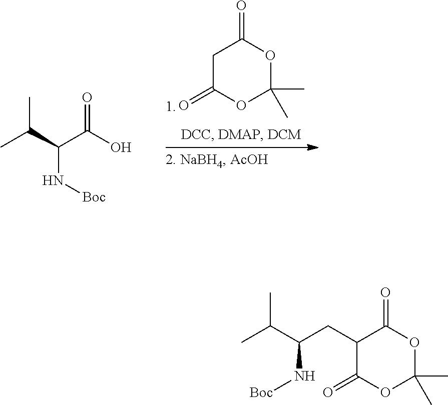

Boc-L-valine (44.9 kg), 2,2-dimethyl-1,3-dioxane-4,6-dione (32.9 kg) and DMAP (35.5 kg) in DCM (607 kg) pre-cooled at −10 to 0° C. were added to a solution of DCC (55.5 kg) in DCM (613 kg) over 3 h and aged for 16 h at −10 to 0° C. 10% citric acid aqueous solution (449 kg) was added whilst maintaining a temperature below 10° C. The resulting slurry was aged for 2 h at 0 to 10° C. then filtered. The filter cake was washed with DCM (91 kg). The filtrate was separated and the organic layer was washed with 10% citric acid aqueous solution (two times 450 kg) and 10% NaCl aqueous solution (449 kg). To organic phase (1200 kg), was added acetic acid (75.0 kg) whilst maintaining a temperature between −10 to 0° C. Sodium Borohydride (18.0 kg) was added in portions over 5 h whilst maintaining a temperature in the range −10 to 0° C. and then resulting mixture was aged at −10 to 0° C. for an additional 16 h. The mixture was warmed to 15 to 25° C., and aged for 2 h. The mixture was then washed with 14% NaCl aqueous solution (450 kg) followed by a second wash with 14% NaCl aqueous solution (432 kg) and a final water wash (444 kg). The organic phase was concentrated under reduced pressure to 2-4 vol. Iso-propanol (143 kg) was added to the residue and concentrated to 4-5 vol. under reduced pressure. After cooling to −10 to 0° C. and aging for 8 h, the resulting slurry was filtered, washed with IPA (38 kg) and dried to afford the title intermediate (46.7 kg, 69% yield) as a white solid.

### Preparation of Intermediate 228

### tert-butyl (R)-2-isopropyl-5-oxopyrrolidine-1-carboxylate

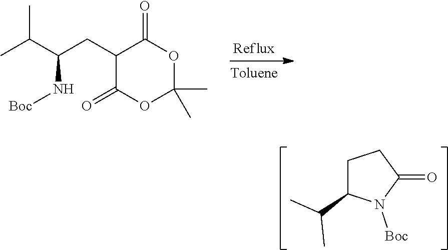

tert-butyl (R)-(1-(2,2-dimethyl-4,6-dioxo-1,3-dioxan-5-yl)-3-methylbutan-2-yl)carbamate (intermediate 227) (46.7 kg) in toluene (333 kg) was heated to reflux and aged for 4 h. The mixture was cooled to ambient temperature, filtered and washed with toluene (20 kg). The combined filtrates were concentrated to dryness at reduced pressure to afford the desired compound (31.05 kg, 96% yield) as an oil which was used directly without further purification.

### Preparation of Intermediate 229

### tert-butyl (5R)-2-hydroxy-5-isopropylpyrrolidine-1-carboxylate

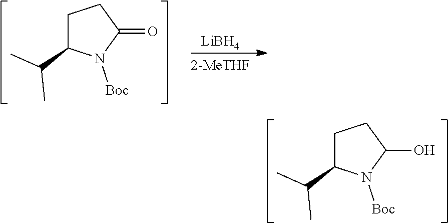

tert-butyl (R)-2-isopropyl-5-oxopyrrolidine-1-carboxylate (intermediate 228) (30.9 kg) in 2-MeTHF (26.7 kg) was cooled to −5 to 5° C. A solution of LiBH₄ in 2-MeTHF (1M, 45.2 kg, 54.4 mol) was added over 3 h and the mixture was aged for 4 h. A cold aqueous solution of 5% NaHCO₃ (163 kg) was added at −5 to 5° C. over 3 h and aged for an additional 2 h. The mixture was warmed to ambient temperature and aged for a further 2 h. The aqueous layer was separated and the organic layer was washed with 10% NaCl aqueous solution (170 kg) and water (155 kg). During the water wash, an emulsion formed and solid NaCl (3.1 kg) was added to affect the separation. After removal of the aqueous layer, the organic layer was concentrated under reduced pressure to dryness to afford the desired compound (28.5 kg, 91% yield) as an oil, which was used directly without further purification.

### Preparation of Intermediate 230

### tert-butyl (R)-(6-((2-methoxyethyl)(methyl)amino)-2-methylhexan-3-yl)carbamate

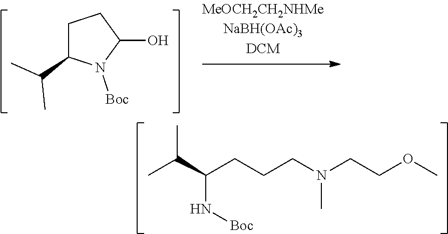

tert-butyl (5R)-2-hydroxy-5-isopropylpyrrolidine-1-carboxylate (intermediate 229) (28.55 kg) in DCM (344 kg), at 15 to 25° C. was treated with 2-methoxy-N-methylethan-1-amine (12.3 kg, 138.0 mol) and the resulting mixture was aged for 1 h. Sodium triacetoxyborohydride (40.12 kg) was added in portions over 5h whilst maintaining a temperature between 15 to 25° C. and the resulting mixture was aged for 48 h. The reaction mixture was quenched by the addition of 8% NaOH aqueous solution (184 kg) over 2 h whilst maintaining a temperature between 15 to 25° C. and the mixture was aged for a further 2 h. The water layer was separated, and the organic layer was washed with water (169 kg). The organic layer was then concentrated under reduced pressure to dryness to afford the title intermediate (33.26 kg, 88% yield) as an oil which was used directly without further purification.

### Preparation of intermediate 231

### (R)—N¹-(2-methoxyethyl)-N¹,5-dimethylhexane-1,4-diamine, dihydrochloride

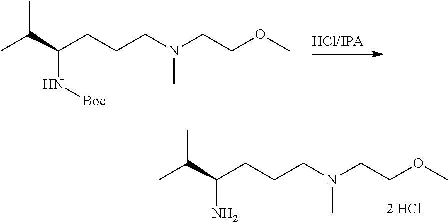

To 4 molar solution of HCl in iso-propanol (84.80 kg) at ambient temperature was added a solution of tert-butyl (R)-(6-((2-methoxyethyl)(methyl)amino)-2-methylhexan-3-yl)carbamate (intermediate 230) (32.38 kg) in iso-propanol (25.6 kg) over 3 h and the mixture was aged at ambient temperature for an additional 19 h. Methyl tert-butyl ether (95.25 kg) was then added over 1 h and the mixture was aged for 2.5 h. The resulting slurry was filtered and washed with MTBE (53 kg). The filter cake was dried to afford the title compound (23.92 kg, 81% yield) as a white solid.

### Preparation of Intermediate 232

### ethyl 1-benzyl-3-(chloromethyl)pyrrolidine-3-carboxylate

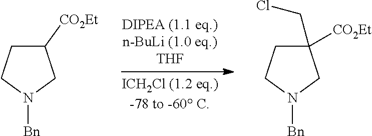 [SMILES: CCOC(=O)C1CCN(Cc2ccccc2)C1 ; CCOC(=O)C1(CCl)CCN(Cc2ccccc2)C1]

To a solution of DIPEA (952 g, 1.1 eq.) in THF (6 L) which was cooled to −35 to −25° C. was added n-BuLi (2.33 kg, 2.5 M in hexane, 1.0 eq.) whilst maintaining a temperature below −25° C. The resulting mixture was aged at −35 to −25° C. for an additional 30 min then cooled to between −78 to −60° C. A solution of ethyl 1-benzylpyrrolidine-3-carboxylate (2 kg, 1.0 eq.) in THF (2 L) at −78 to −60° C. was added and stirred for an addition 30 min. Chloroiodomethane (1.81 kg, 1.2 eq.) was then charged at −78 to −60° C. The reaction mixture was aged at −60 to −40° C. for 2 h. To the reaction mixture was added to citric acid aqueous solution (660 g in 6 L H₂O) at a temperature between 0 to 10° C. and the resulting mixture was aged at 20 to 30° C. for an additional 20 min. After separating the layers, the aqueous layer was extracted with EtOAc (6 L) and the combined organic layers washed with brine (6 L) then warmed to 50 to 60° C. Oxalic acid (2.22 kg) was charged at 50 to 60° C. The resulting mixture was stirred at 50 to 60° C. for 3 h then cooled to 20 to 30° C. and aged overnight. The resulting solid was filtered and the cake was washed with ethyl acetate (2 L). The wet cake was added to toluene (4 L), H₂O (8 L) and K₃PO₄ (1.5 eq.) and the resulting mixture was aged at 20 to 30° C. for 20 min. After separating the layers, the aqueous layer was extracted with toluene (2 L). The organic layers were combined and washed twice with water (2 L). The organic phase was concentrated under reduced pressure to afford 4.2 kg of the desired compound as a toluene solution (46 wt % by assay, giving an assay yield of 80%).

### Preparation of Intermediate 233

### 1-benzyl-3-(chloromethyl)pyrrolidine-3-carbaldehyde

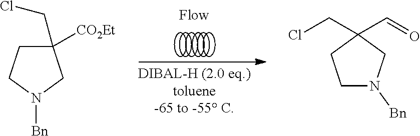 [SMILES: CCOC(=O)C1(CCl)CCN(Cc2ccccc2)C1 ; O=CC1(CCl)CCN(Cc2ccccc2)C1]

Reaction conducted in a flow chemistry system: A solution of ethyl 1-benzyl-3-(chloromethyl)pyrrolidine-3-carboxylate (intermediate 232) (4.4 kg) in toluene (26 L) was pumped at 26.7 mL/min and cooled to −60° C. After cooling, it was then mixed with a cooled solution of DIBAL-H (28.1 mol) in toluene at −60° C. (28 L) with a pumping rate of 32.1 mL/min. The mixture was passed through a Perfluoroalkoxy (PFA) coil tube reactor at −60° C. (total flow rate of 58.8 mL/min with a residence time of 5 seconds). The resulting mixture was mixed with cooled MeOH (−60° C.) which was pumped at the rate of 15.2 mL/min. This mixed solution was pumped to another PFA coil tube reactor at −60° C. (total flow rate of 74 mL/min with a residence time of 5 seconds). The resulting mixture was collected into a receiver which contained 20 wt % aq. solution Rochelle's salt (20 V). The layers were separated, and the organic phase was twice washed with water (2×44 L). The organic phase was combined with another 3.0 kg batch prepared in an analogous manner and concentrated under reduced pressure to afford 20.8 kg of a toluene solution of the desired compound (25.5 wt % assay by HPLC, giving an assay yield of 85%) which was used directly without further purification.

¹H NMR (300 MHz, Chloroform-d): δ 9.62 (s, 1H), 7.39-7.20 (m, 5H), 3.83-3.57 (m, 4H), 2.96 (d, J=10.2 Hz, 1H), 2.80-2.55 (m, 3H), 2.17 (ddd, J=13.9, 7.9, 6.1 Hz, 1H), 1.83 (ddd, J=13.4, 7.8, 5.5 Hz, 1H).

### Preparation of Intermediate 234

### (R)-4-(6-benzyl-2,6-diazaspiro[3.4]octan-2-yl)-N-(2-methoxyethyl)-N,5-dimethylhexan-1-amine

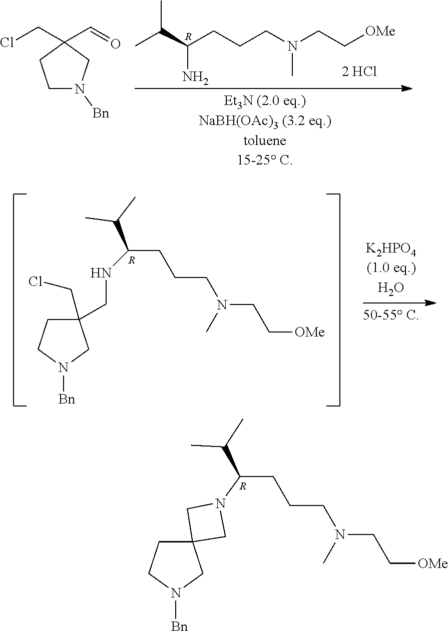

To a solution of 1-benzyl-3-(chloromethyl)pyrrolidine-3-carbaldehyde (intermediate 233) in toluene (3.0 kg, 10 wt %) diluted with toluene (30 L) and (R)—N¹-(2-methoxyethyl)-N¹,5-dimethylhexane-1,4-diamine, dihydrochloride (intermediate 231) (3.47 kg) was added triethylamine (2.55 kg, 25.2 mol) at 20 to 30° C. The resulting mixture was aged for 2 h at 20 to 30° C. Then sodium triacetoxyborohydride (9.0 kg) was charged at 20 to 30° C. and the mixture was aged for 12 h. The reaction mixture was cooled to 5 to 15° C. and 25 wt % NaOH aqueous solution (25 L, ˜16.75 eq.) was added maintaining a temperature below 35° C. The resulting mixture was aged at 20 to 30° C. for 25 mins and the layers were separated. The organic layer was washed with 15 wt % aq. NaCl (10 L) and the layers were again separated and water (18 L) was charged to the organic phase. The pH of the aqueous phase was adjusted to 6-7 with 4M aq. HCl whilst maintaining an internal temperature below 35° C. The organic phase was then discarded and the aqueous phase was separated and basified to pH 8-9 with K₂HPO₄.

The resulting mixture was warmed to 50 to 55° C. and aged for 3 h. The reaction mixture was then cooled to ambient temperature and combined with other two batches (2.4 kg+3.0 kg). The combined streams were washed with methyl tert-butyl ether three times (3×40 L). To the resulting aqueous layer was added additional methyl tert-butyl ether (83 L) and the aqueous phase was basified to pH 9-10 using 8 wt % aq. NaOH whilst maintaining a temperature between 15 to 35° C. The aqueous layer was separated, and the organic layer was washed with three times water (3×30 L). The organic layer was then concentrated under reduced pressure to approximately 3 volumes and then flushed with methanol three times (3×30 L) and concentrated to dryness to afford the desired intermediate (12.4 kg, 90% isolated yield) as light-yellow oil, which was used directly without further purification.

### Preparation of Intermediate 234a (Citric Acid Salt of Intermediate 234)

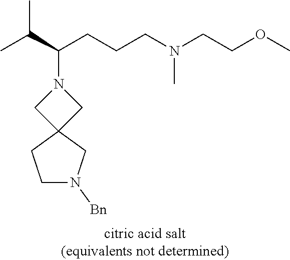 [SMILES: COCCN(C)CCC[C@H](C(C)C)N1CC2(CCN(Cc3ccccc3)C2)C1]

EtOH (80 ml) and intermediate 234 (20 g) were added in a round bottom flask. Next, a 0.5 M solution of citric acid in EtOH (100 ml; 1 equivalent) was added to the mixture in the round bottom flask at room temperature. Subsequently, the mixture was evaporated till dryness (Rotavap, 40° C.). Acetonitrile (200 ml) was added to the residue and the mixture was evaporated till dryness (Rotavap, 40° C.). Acetonitrile (100 ml) was added to the residue and stirred overnight on a magnetic heating plate at room temperature. Finally, intermediate 234a was filtered off and dried at room temperature.

### Preparation of Crystalline Form of Citric Acid Salt of Intermediate 234 (Intermediate 234b)

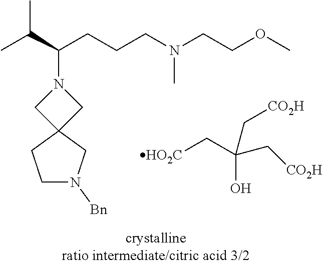

Intermediate 234a (3.72 g) was added to acetonitrile (20 ml) at room temperature and the mixture was stirred. The mixture was heated to 60° C. until the reaction mixture became homogeneous (about 10 minutes). Next, the mixture was cooled to 50° C. at a rate of 0.5° C./min. Next, seeds were added (19 mg of intermediate 234a; 0.5 w/w %) and the mixture was aged while stirring during 3 hours and 30 minutes. Next, the mixture was cooled non-linear to 20° C. over 8 hours with an exponent of 2,3. The obtained mixture was stirred overnight and the product was filtered off and dried (overnight at room temperature in hood). After isolation, intermediate 234b was obtained (2.75 g; yield 73.9%) as the crystalline form of the citric acid salt of intermediate 234. The obtained ratio of the intermediate/citric acid is 3/2 (NMR).

The non-linear cooling referred to above was done according to the formula below:

A new linear ramp is started every 30 seconds during the defined duration of the cooling. The ramp is calculated according to the following equation:

$${T}_{set} = {T}_{startvalue} - [ ( {T}_{startvalue} - {T}_{endvalue} ) * {( \frac{{t}_{action} + 30 s}{Duration} )}^{n} ]$$

Tₛₑₜ: Set value for each new ramp
T_{start value}: Measured mixture temperature at the start of the cooling trajectory
T_{end value}: Defined end value of cooling trajectory
t_{action}: Actual time from the start of the cooling
Duration: Defined cooling duration
n: Exponent

¹H NMR (400 MHz, MeOH-d₄) δ ppm 0.91 (3H, d, J=6.88 Hz) 0.98 (3H, d, J=6.88 Hz) 1.46-1.57 (2H, m) 1.67-1.87 (2H, m) 1.94-2.03 (1H, m) 2.20-2.29 (2H, m) 2.62-2.69 (2H, m) 2.72-2.77 (4H, m) 2.77-2.82 (2H, m) 2.90 (2H, t, J=7.32 Hz) 2.95-3.02 (2H, m) 3.07-3.16 (2H, m) 3.16-3.22 (2H, m) 3.37 (3H, s) 3.68-3.72 (2H, m) 3.83-3.89 (2H, m) 3.90-3.92 (2H, m) 3.94-4.06 (2H, m) 7.32-7.43 (5H, m)

### Preparation of Intermediate 224

### (R)—N-(2-methoxyethyl)-N,5-dimethyl-4-(2,6-diazaspiro[3.4]octan-2-yl)hexan-1-amine

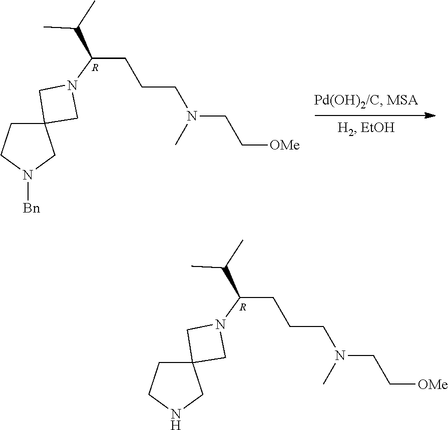

To palladium hydroxide on carbon (1.2 kg) in EtOH (1.47 kg) cooled to −5 to 5° C. were added methanesulfonic acid (MSA) (11 kg), (R)-4-(6-benzyl-2,6-diazaspiro[3.4]octan-2-yl-N-(2-methoxyethyl)-N,5-dimethylhexan-1-amine (intermediate 234) (10 kg) and EtOH (250 L). The mixture was warmed to 35-45° C. and stirred under a hydrogen atmosphere (0.27 to 0.40 MPa) for 16-20 h. The mixture was filtered over diatomite (20 kg) and the pad was washed with EtOH (24 L). The filtrate was concentrated under reduced pressure (<40° C.) to 2˜3 vol. and then flushed twice with 2-MeTHF (73 kg and 47 kg) to give a 2˜3 vol. solution. After dilution with 2-MeTHF (65 kg), 10% aq. sodium sulfate (30 kg) was added and the mixture was cooled to 0 to 10° C., followed by the addition of 16% aq. NaOH (50 kg) to adjust the pH to 13-14. The temperature was adjusted to 15 to 25° C. and stirred for 30 to 60 min. The aqueous layer was separated and extracted twice with 2-MeTHF (47 kg×2). The combined organic layers were concentrated under reduced pressure (<40° C.) to 3-4 vol. and 2-MeTHF (950 g) was added. After concentration under reduced pressure (<40° C.) to 3-4 vol., the resulting solution was diluted with 2-MeTHF (30 kg), dried by passing through 4A molecular sieves (25 kg) and washed with 2-MeTHF (30 kg). The final solution was concentrated to afford the desired compound (6.7 kg) as an oil with 90.1% assay purity in a 79% corrected yield.

### Preparation of Intermediate 225

### (R)-4-(6-(3,6-dichloro-1,2,4-triazin-5-yl)-2,6-diazaspiro[3.4]octan-2-yl)-N-(2-methoxyethyl)-N,5-dimethylhexan-1-amine

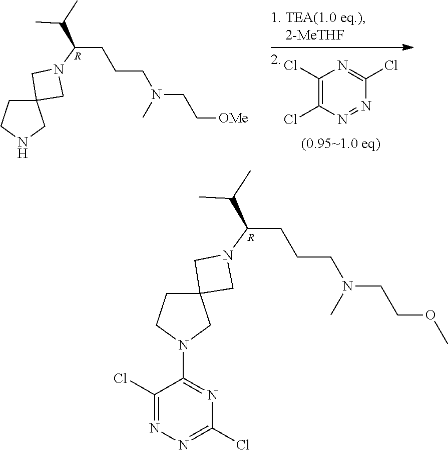

To (R)—N-(2-methoxyethyl)-N,5-dimethyl-4-(2,6-diazaspiro[3.4]octan-2-yl)hexan-1-amine (intermediate 224) (100 g) was added 2-MeTHF (430 g) and TEA (68 g) and the mixture was cooled to −50 to −40° C. 3,5,6-trichloro-1,2,4-triazine (62 g) in 2-MeTHF (172 g) was added and the mixture was stirred for 1 to 3 h. The resulting mixture was warmed to −20 to −10° C. and a 7% NaHCO₃ aqueous solution was added, the mixture was warmed to 20 to 30° C. and stirred for 30 to 60 min. The aqueous layer was removed and the organic layer was washed with 10% Na₂SO₄ (500 g). The organic layer was dried by passing through 4A molecular sieves (220 g) and washed with 2-MeTHF (180 g). The title intermediate was afforded in 90% assay yield as a solution 14.8 wt % in 2-MeTHF.

### Compound 393

### (R)-2-((3-chloro-5-(2-(6-((2-methoxyethyl)(methyl)amino)-2-methylhexan-3-yl)-2,6-diazaspiro[3.4]octan-6-yl)-1,2,4-triazin-6-yl)oxy)-N-ethyl-5-fluoro-N-isopropylbenzamide

### Synthesis Method A for Compound 393

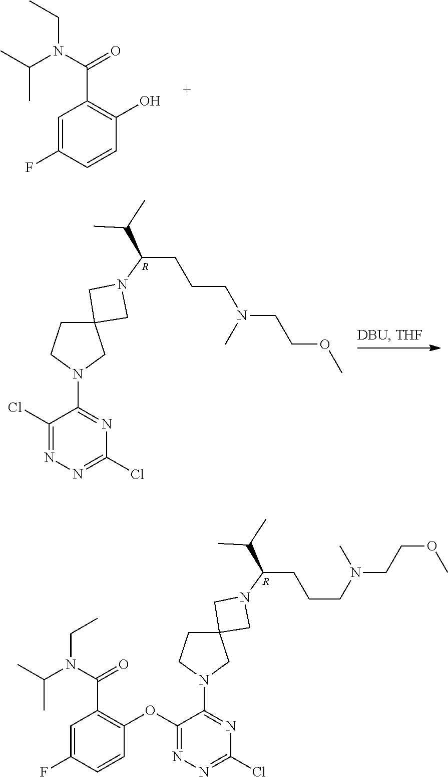

The mixture of N-ethyl-5-fluoro-2-hydroxy-N-isopropylbenzamide (intermediate 28) (1.10 g, 4.88 mmol), (R)-4-(6-(3,6-dichloro-1,2,4-triazin-5-yl)-2,6-diazaspiro[3.4]octan-2-yl)-N-(2-methoxyethyl)-N,5-dimethylhexan-1-amine (intermediate 225) (1.70 g, 3.82 mmol) and DBU (750 mg, 4.93 mmol) in anhydrous THF (15 mL) was stirred at 40° C. for 8 h. After cooled to RT, the mixture was concentrated under reduced pressure, the resulting residue was diluted with DCM (60 mL) and washed with H₂O (20 mL×3). The organic layer was dried over anhydrous Na₂SO₄, filtered and concentrated under reduced pressure to give the crude product which was purified FCC (MeOH/DCM=0% to 10%) to afford a yellow oil (1.40 g), which was further separated by SFC over DAICEL CHIRALPAK AD (column: 250×50 mm, 10 um; Mobile phase: A: Supercritical CO₂, B: EtOH (0.1% ammonia), A:B=50:50 at 70 mL/min; Column Temp: 38° C.; Nozzle Pressure: 100 Bar; Nozzle Temp: 60° C.; Evaporator Temp: 20° C.; Trimmer Temp: 25° C.; Wavelength: 220 nm) to afford the title compound (1.0 g).

### Synthesis Method A for Compound 393

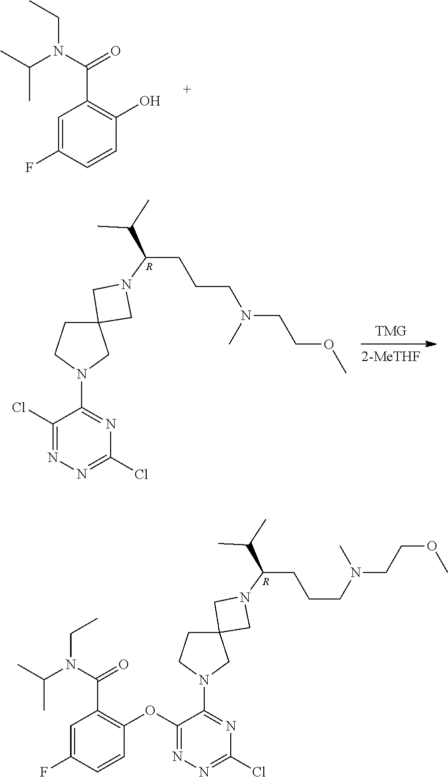

To a 2-MeTHF solution of (R)-4-(6-(3,6-dichloro-1,2,4-triazin-5-yl)-2,6-diazaspiro[3.4]octan-2-yl)-N-(2-methoxyethyl)-N,5-dimethylhexan-1-amine (intermediate 225) (676 g of a 14.8 wt % solution in 2-MeTHF, 100 g corrected of intermediate 225) and N-ethyl-5-fluoro-2-hydroxy-N-isopropylbenzamide (intermediate 28) (50.6 g) in 2-MeTHF (40 g) at 20 to 30° C. was added tetramethylguanidine (31 g) and the mixture was stirred for 40 to 48 h. A 7% NaHCO₃ aqueous solution (500 g) was added and the mixture was stirred for 30 to 60 min. The aqueous layer was removed and the organic layer was washed with twice with 4% NaOH aqueous solution (2×500 g) and once with 10% Na₂SO₄ aqueous solution (500 g). The organic layer was concentrated under reduced pressure (<40° C.) to 2.2-3.0 vol. and flushed three times with MeOH (1×790 g and 2×395 g) until both 2-MeTHF and water content were both ≤1.0% to afford the desired compound in 86% assay yield as a 60.1 wt % solution in methanol.

### Compound A

### (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide

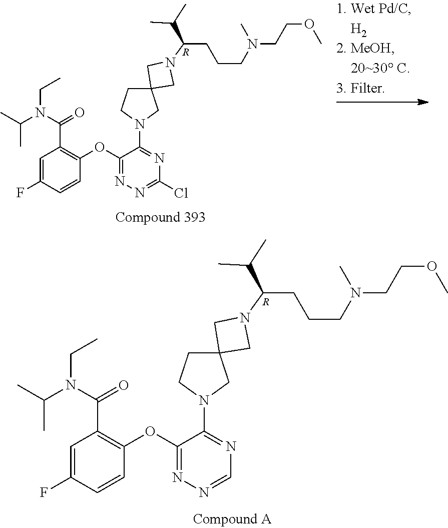

A methanol solution of (R)-2-((3-chloro-5-(2-(6-((2-methoxyethyl)(methyl)amino)-2-methylhexan-3-yl)-2,6-diazaspiro[3.4]octan-6-yl)-1,2,4-triazin-6-yl)oxy-N-ethyl-5-fluoro-N-isopropylbenzamide (Compound 393) (163.93 g of a 60.1 wt % solution in MeOH, 100 g corrected of Compound 393), palladium on carbon (10 g) and MeOH (316 g) was stirred at 20 to 30° C. under a hydrogen atmosphere (0.20 to 0.30 Mpa) for 18 h. The mixture was filtered over diatomite (75 g) and the cake was washed with MeOH (158 g). The filtrate was concentrated under reduced pressure (≤40° C.) to ˜3 vol., then flushed with isopropyl acetate (IPAc, 870 g) concentrating to ˜3 vol. The mixture was then diluted with IPAc (696 g) and a 20% Na₂CO₃ aqueous solution was added (500 g). The mixture was stirred for 30 to 60 min. The aqueous layer was removed. The organic layer was washed with water (500 g) then concentrated under reduced pressure <45° C. to ˜3 vol. The title intermediate was afforded in approximately 90% assay yield as a 48.1 wt % solution in IPAc.

### Example 4—Synthesis of (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide oxalate (Compound A3)

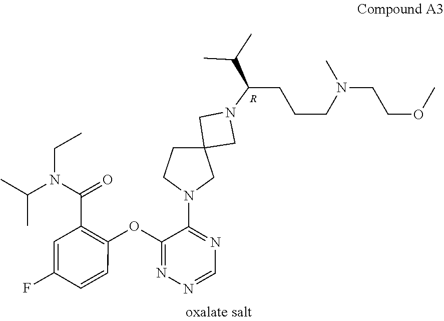

To a solution of (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A) (270 mg, 0.450 mmol) in 20 mL of ACN (20 mL) was added oxalic acid (81.0 mg, 0.900 mmol). After addition, the reaction mixture was stirred at RT for 1 h. Then the reaction mixture was concentrated, the residue was re-dissolved in ACN and deionized water, and lyophilized to afford the title compound (350 mg) as white solid.

¹H NMR (400 MHz, Methanol-d₄): δ=8.48 (s, 1H), 7.52-7.11 (m, 3H), 4.54-3.64 (m, 12H), 3.40-3.34 (m, 5H), 3.23-3.13 (m, 2H), 2.90 (s, 3H), 2.54-2.27 (m, 2H), 2.19-2.03 (m, 1H), 1.97-1.77 (m, 2H), 1.75-1.50 (m, 2H), 1.35-0.65 (m, 17H).

¹H NMR (400 MHz, DMSO-d₆): δ=8.51 (s, 1H), 7.51-7.29 (m, 3H), 4.29-3.34 (m, 12H), 3.23-2.84 (m, 7H), 2.70 (s, 3H), 2.35-2.09 (m, 2H), 2.05-1.85 (m, 1H), 1.81-1.58 (m, 2H), 1.56-1.33 (m, 2H), 1.18-0.60 (m, 17H).

LCMS (ESI) (Method 2): Rₜ=1.969 min, m/z found 600.4 [M+H]⁺.

### Example 5—Synthesis of Compound A1

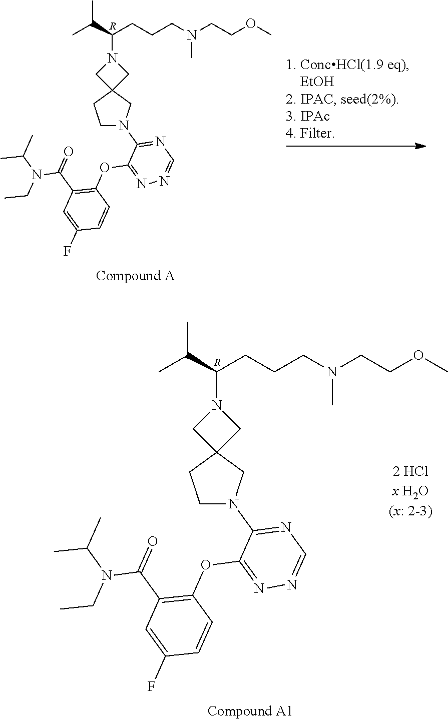

To a solution of Compound A (207.90 g of a 48 wt % solution in IPAc, 100 g of active Compound A) in IPAc (360 g) was added EtOH (63 g) at 20 to 25° C. The solution was then treated with conc. HCl (32.9 g) in EtOH (49.5 g) over ˜15 min. The mixture was seeded with crystalline Compound A1 seed (2 g, 2% seed load) then aged for 18 h. IPAc (870 g) was added slowly over 4 h at between 20 to 25° C. and the slurry was stirred for an additional 18 h. After cooling to −5° C., the product was filtered, washed with IPAc (522 g) and dried under vac at 20-30° C. to afford the weakly crystalline Compound A1 as a white solid (91.0% yield, 115.4 g). (Note: A small amount of seed material used in the reaction was obtained via an analogous reaction protocol on small-scale.)

Recrystallisation: A solution of weakly crystalline Compound A1 (100 g), EtOH (166 g), purified water (21.5 g) and IPAc (178 g) was stirred at 20 to 30° C. for 0.5-2 h to get a clear solution. Extra IPAc (522 g) was added dropwise over 1-2 h, and then the mixture was seeded with crystalline Compound A1 seed (2 g, 2% seed load). Then the mixture was aged for 18-20 h, IPAc (348 g) was added slowly over 12 h at between 20 to 30° C., and the slurry was stirred for an additional 55-60 h. The product was filtered, washed with IPAc (158 g) and dried in vacuo at 20-30° C. to afford Compound A1 as a white solid (85% yield, 85.0 g, net).

¹HNMR (DMSO-d₆, 400 MHz): δ=11.60 (1H, brs), 10.8 (1H, brs), 8.52 (1H, s), 7.36 (3H, m), 3.97-4.20 (7H, m), 3.64-3.71 (4H, m), 3.47 (7H, m), 3.25 (2H, m), 3.05 (3H, m), 2.73 (3H, s), 2.10-2.45 (1H, m), 1.99 (1H, m), 1.78 (2H, m), 1.55 (2H, m), 0.83-1.12 (12H, m), 0.70 (2H, m).

LCMS (Method 7): Rₜ=0.669 min, m/z found 600.5 [M+H]⁺.

### Example 6—Synthesis of crystalline form A of (R)—N-ethyl-5-fluoro-N-isopropyl-2-((5-(2-(6-((2-methoxyethyl)(methyl)amino)-2-mthylhexan-3-yl)-2,6-diazaspiro[3.4]octan-6-yl)-1,2,4-triazin-6-yl)oxy)benzamide bis-besylate salt hydrate (Compound A4) (equivalent water not determined)

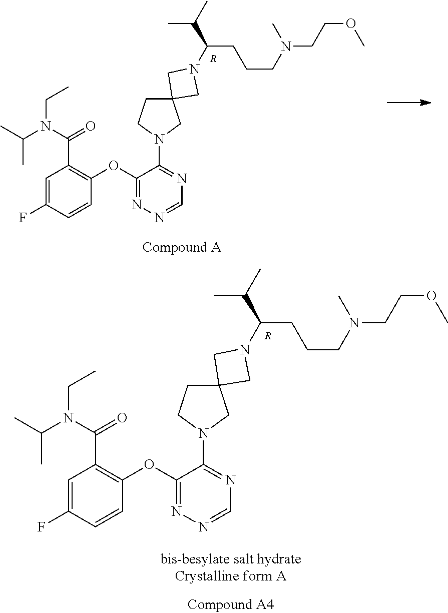

43.06 g benzenesulfonic acid (2 equivalents with respect to the free base Compound A) was added to 840 ml of an acetone/water 95/5 v/v mixture and dissolved. 192.8 g of a solution of Compound A (containing 80 g API) in IPAc was added. The material was dissolved, resulting in a clear solution. A further 80 ml of IPAc is added and the temperature was adjusted to 25° C. 2% of seeds were added and the mixture was stirred for an hour at 25° C. Then 28.8 V (2312 ml) of IPAc was added over a period of 8 hours. Afterwards the suspension was stirred for 18 hours at 25° C. The suspension was filtered and washed with 320 ml of a mixture of acetone/water/IPAc 23.75/1.75/75 v/v/v. 122.91 g of crystalline form A bis-besylate hydrate (equivalent water not determined) was obtained.

A skilled person will understand that a small amount of initial seed material used in the reaction above can be obtained via an analogous reaction protocol on small-scale without addition of seeds and wait for spontaneous nucleation.

Initial seeds of the besylate salt were also obtained during salt screening experiments. In these experiments 100 mg of the free base was weighed into 2 mL vials, and then 200 μL of ethyl acetate or acetone was added to dissolve the free base. 1 eq counter-ions (benzenesulfonic acid) were added to the samples, and the samples were stirred at 25° C. for 3 days. The suspension obtained was centrifuged and yielded initial seeds.

An appropriate amount of crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate was dissolved in deuterated DMSO and the 1D ¹H NMR spectrum was recorded.

A Bruker AVANCE NEO-600 MHz NMR spectrometer equipped with a Bruker 5 mm PA BBO 600S3 BB-H-D-05 Z-GRD high resolution probe and running TOPSPIN 4.0 software, was used to collect a 1-dimensional proton experiment at 300K on the sample in deuterated DMSO.

¹H NMR (600 MHz, DMSO-d₆) δ ppm 0.69 (br s, 2H) 0.82-0.98 (m, 9H) 1.07 (br s, 4H) 1.31-1.46 (m, 1H) 1.51 (br d, J=2.91 Hz, 1H) 1.69 (br d, J=3.45 Hz, 2H) 1.98 (br s, 1H) 2.06-2.45 (m, 2H) 2.77 (br s, 3H) 2.87-3.19 (m, 3H) 3.24 (br s, 1H) 3.31 (s, 6H) 3.64 (br s, 4H) 3.71-4.59 (m, 7H) 7.24-7.54 (m, 9H) 7.61 (br d, J=7.27 Hz, 4H) 8.45-8.60 (m, 1H) 9.24 (br s, 1H) 9.44-9.82 (m, 1H).

### Example 7—Alternative synthesis of crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate (Compound A4) (equivalent water not determined)

A mixture of isopropanol/water 95/5 (24 ml) was charged in a flask and heated to 40° C. Benzenesulfonic acid (4.31 g; 98%) was added. Subsequently, 19.3 g of a solution of Compound A (containing 8 g of Compound A) in IPAc was added. Another 16 ml of IPAc was added. 2% of seeds were added and the mixture was stirred for 1 hour at 40° C. Then IPAc was added (115.2 ml) dropwise over a period of 8 hours. Next, the mixture was cooled to 0° C. for 15 hours. The suspension was filtered and the wet cake was washed with (IPA/H₂O 95/5)/IPAc 1/6 (32 ml). The wet cake was dried at 25° C. for 16 hours to obtain 11.44 g of crystalline form A bis-besylate hydrate (equivalent water not determined).

In the examples, Compound A4 is a Compound covered by claim 1. The other Compounds in the examples are for illustrative purposes. Some intermediates (for example intermediate 234b) are claimed intermediates.

### Analytical Methods Used in the Experimental Part Above

The analytical information in the Compounds above, was generated by using the analytical methods described below.

### NMR-Methods

Some NMR experiments were carried out using a Bruker Avance III 400 spectrometer at ambient temperature (298.6 K), using internal deuterium lock and equipped with BBO 400 MHz S1 5 mm probe head with z gradients and operating at 400 MHz for the proton and 100 MHz for carbon. Chemical shifts (δ) are reported in parts per million (ppm). J values are expressed in Hz.

Some NMR experiments were carried out using a Varian 400-MR spectrometer at ambient temperature (298.6 K), using internal deuterium lock and equipped with Varian 400 4NUC PFG probe head with z gradients and operating at 400 MHz for the proton and 100 MHz for carbon. Chemical shifts (δ) are reported in parts per million (ppm). J values are expressed in Hz.

Some NMR experiments were carried out using a Varian 400-VNMRS spectrometer at ambient temperature (298.6 K), using internal deuterium lock and equipped with Varian 400 ASW PFG probe head with z gradients and operating at 400 MHz for the proton and 100 MHz for carbon. Chemical shifts (6) are reported in parts per million (ppm). J values are expressed in Hz.

Some NMR experiments were carried out using a Bruker AVANCE III HD 300 spectrometer at ambient temperature (298.6 K), using internal deuterion lock and equipped with PA BBO 300S1 BBF-H-D-05 Z 5 mm probe head with z gradients and operating at 300 MHz for the proton and 75 MHz for carbon. Chemical shifts (d) are reported in parts per million (ppm). J values are expressed in Hz.

### LCMS (Liquid Chromatography/Mass Spectrometry)

### General Procedure

The High Performance Liquid Chromatography (HPLC) measurement was performed using a LC pump, a diode-array (DAD) or a UV detector and a column as specified in the respective methods. If necessary, additional detectors were included (see Table 2 below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc.,). For molecules with multiple isotopic patterns (Br, Cl), the reported value is the one obtained for the lowest isotope mass. All results were obtained with experimental uncertainties that are commonly associated with the method used.

Hereinafter, “SQD” means Single Quadrupole Detector, “RT” room temperature, “BEH” bridged ethylsiloxane/silica hybrid, “HSS” High Strength Silica, “DAD” Diode Array Detector.

TABLE 2
LCMS Method Codes (Flow expressed in mL/min; column
temperature (T) in ° C.; Run time in minutes).

| Method | | | | | | | Flow | Run |
|---|---|---|---|---|---|---|---|---|
| code | Instrument | Column | Mobile phase | Gradient | | | Column T | time |
| 1 | Agilent | Waters | mobile phase | 100% A was held for 1 min, A | | | 0.8 | 10 |
| | | XBridge | A: H₂O with | gradient from 100% A to 40% A | | | 50 | |
| | | C18 (2.0 × | 0.04% TFA; | is applied in 4 min, and 40% A | | | | |
| | | 50 mm, | mobile phase | down to 15% A in 2.5 min. And | | | | |
| | | 5 uM) | B: ACN with | then return to 100% A in 2 | | | | |
| | | | 0.02% TFA | min and held for 0.5 min. | | | | |
| | | | | The post time is 0.5 min. | | | | |
| 2 | Agilent | Waters | mobile phase | First, 90% A was held for 0.8 | | | 0.8 | 10 |
| | | XBridge | A: H₂O with | min. Then a gradient was | | | 50 | |
| | | C18 (2.0 × | 0.04% TFA; | applied to 20% A and 80% B in | | | | |
| | | 50 mm, | mobile phase | 3.7 min and held for 3 min. | | | | |
| | | 5 um) | B: ACN with | And then return to 90% A in | | | | |
| | | | 0.02% TFA | 2 min and held for 0.5 min. | | | | |
| | | | | The post time is 0.5 min. | | | | |
| | | | | Time (min) | A % | B % | | |
| 7 | Agilent LC | XBridge | Mobile phase | Initial | 95 | 5 | 1.5 | 20 |
| | 1260 with | C18, 4.6 × | A 0.05% TFA | 11.0 | 65 | 35 | 45 | |
| | MS6120 | 150 mm, | in H₂O | 13.0 | 5 | 95 | | |
| | | 3.5 μm | Mobile phase | 15.0 | 5 | 95 | | |
| | | | B 0.05% TFA | 16.0 | 95 | 5 | | |
| | | | in ACN | 20.0 | 95 | 5 | | |

### Analytical SFC

### General Procedure for SFC Methods

The SFC measurement was performed using an Analytical Supercritical fluid chromatography (SFC) system composed by a binary pump for delivering carbon dioxide (CO₂) and modifier, an autosampler, a column oven, a diode array detector equipped with a high-pressure flow ccli standing up to 400 bars. Analytical SFC details are provided below in Table 3. If configured with a Mass Spectrometer (MS) the flow from the column was brought to the (MS). It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

TABLE 3
Analytical SFC Methods (Flow expressed in mL/min; column
temperature (T) in ° C.; Run time in minutes, Backpressure
(BPR) in bars or pound-force per square inch (psi). “ACN”
means acetonitrile; “MeOH” means methanol; “EtOH”
means ethanol; “DEA” means diethylamine. All other
abbreviations used in Table below are as defined before)

| | | | | | Run |
|---|---|---|---|---|---|
| Method | | | | Flow | time |
| code | column | mobile phase | gradient | Col T | BPR |
| 11 | Waters UPCC | A: | from 5% to | 2.8 | 8 |
| | with PDA | Supercritical | 40% of B in | 35 | 1500 |
| | (Chiralpak | CO₂ B: | 4 min and hold | | psi. |
| | IG-3 100 × | EtOH (0.05% | 40% for 2.5 | | |
| | 4.6 mm I.D., | DEA) | min, then 5% | | |
| | 3 um) | | of B for 1.5 | | |
| | | | min | | |

### Crystalline form Intermediate 234b

Crystalline form intermediate 234b may be characterised by an X-ray powder diffraction pattern.

X-ray powder diffraction (XRPD) analysis was carried out on a PANalytical Aeris diffractometer. The instrument is equipped with a Cu-Kα X-ray tube using iCore and dCore tunable optics for the incident and the diffracted beam, respectively. The compound was loaded into the cavity of a 16 mm sample holder using the back loading technique.

Samples were run on XRPD using the method below:
Tube: Cu: K-Alpha (λ=1.541874 Å)
Generator: Voltage: 45 kV; Current: 15 mA
Geometry: Bragg-Brentano
Scan mode: Continuous Scan
Scan Range: 4 to 50 deg.
Step size: 0.0217 deg.
Counting time: 58s
Spinner revolution time: 1 sec
Incident beam path (iCore)
Divergence slit: 1/4°
Soller slit: 0.04 rad
Mask 1: 9 mm
Diffracted beam path (dCore)
Anti scatter slit: 9 mm
Irradiated length: 10 mm
Soller slit: 0.04 rad
Detector: PIXcel3D-Medipix3 1×1

One skilled in the art will recognize that diffraction patterns and peak positions are typically substantially independent of the diffractometer used and whether a specific calibration method is utilized. Typically, the peak positions may differ by about ±0.2° two theta, or less. The intensities (and relative intensities) of each specific diffraction peak may also vary as a function of various factors, including, but not limited to particle size, orientation, sample purity, etc.

The X-ray powder diffraction pattern comprises peaks at 5.82, 10.09 and 18.42 degrees two theta±0.2 degrees two theta.

The X-ray powder diffraction pattern comprises peaks at 5.82, 8.52, 9.20, 10.09, 11.43, 13.61, 14.94, 15.89, 17.03 and 18.42 degrees two theta±0.2 degrees two theta.

Intermediate 234b may further be characterized by an X-ray powder diffraction pattern having four, five, six, seven, eight, nine or more peaks selected from those peaks.

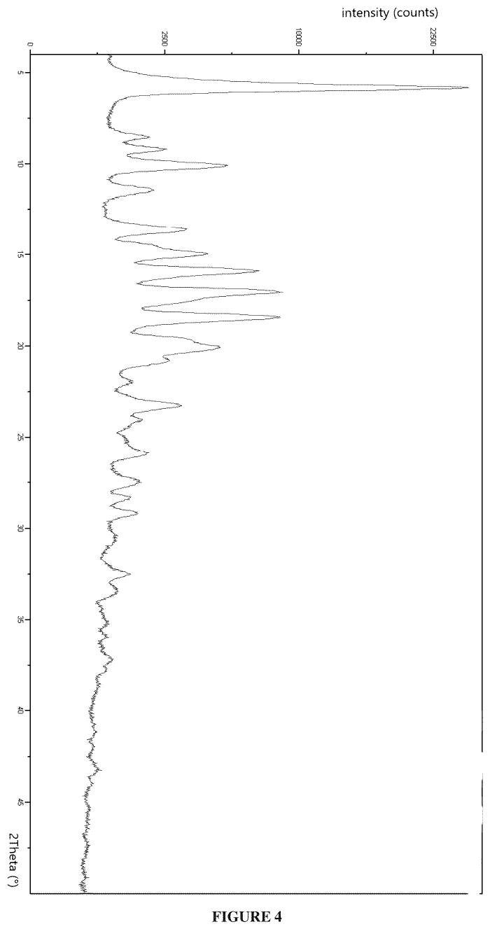

Intermediate 234b may further be characterized by an X-ray powder diffraction pattern substantially as depicted in FIG. 4.

### Crystalline Form A

Crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate may be characterised by an X-ray powder diffraction pattern.

X-ray powder diffraction (XRPD) analysis was carried out on a PANalytical Empyrean diffractometer. The instrument is equipped with a Cu-Kα X-ray tube using iCore and dCore tunable optics for the incident and the diffracted beam, respectively. The compound was loaded into the cavity of a 16 mm sample holder using the back loading technique.

Samples were run on XRPD using the method below:
Tube: Cu: K-Alpha (λ=1.541874 Å)
Generator: Voltage: 45 kV; Current: 40 mA
Geometry: Bragg-Brentano
Scan mode: Continuous Scan
Scan Range: 3 to 35 deg.
Step size: 0.0131 deg.
Counting time: 30s
Spinner revolution time: 1 sec
Incident beam path (iCore)
Program. divergence slit: automatic
Irradiated length: 10 mm
Soller slit: 0.03 rad
Mask 1: 14 mm
Mask 2: 6 mm
Width: 7.7 mm
Diffracted beam path (dCore)
Anti scatter slit: automatic
Irradiated length: 10 mm
Soller slit: 0.04 rad
Detector: PIXcel3D-Medipix3 1×1

One skilled in the art will recognize that diffraction patterns and peak positions are typically substantially independent of the diffractometer used and whether a specific calibration method is utilized. Typically, the peak positions may differ by about ±0.2° two theta, or less. The intensities (and relative intensities) of each specific diffraction peak may also vary as a function of various factors, including, but not limited to particle size, orientation, sample purity, etc.

The X-ray powder diffraction pattern comprises peaks at 5.4, 7.2, 11.1, 11.9 and 21.7 degrees two theta±0.2 degrees two theta. The X-ray powder diffraction pattern may further comprise at least one peak selected from 13.7, 14.5, 14.7, 15.0, 16.5, 17.8, 19.0, 19.4, 20.1 degrees two theta±0.2 degrees two theta.

Form A may further be characterized by an X-ray powder diffraction pattern having four, five, six, seven, eight, nine or more peaks selected from those peaks identified in Table 4.

Form A may further be characterized by an X-ray powder diffraction pattern comprising those peaks identified in Table 4, wherein the relative intensity of the peaks is greater than about 2%, preferably greater than about 5%, more preferably greater than about 10%, more preferably greater than about 15%. However, a skilled person will realize that the relative intensity of the peaks may vary between different samples and different measurements on the same sample.

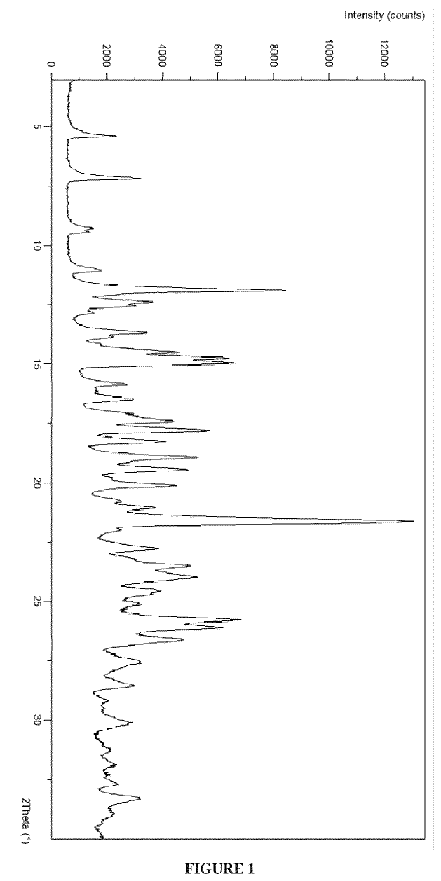

Form A may further be characterized by an X-ray powder diffraction pattern substantially as depicted in FIG. 1.

Table 4 provides peak listings and relative intensity for the XRPD of Crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate (FIG. 1).

TABLE 4

| Pos. [°2Th.] | Rel. Int. [%] |
|---|---|
| 5.3965 | 16.30 |
| 7.1906 | 23.69 |
| 9.2513 | 8.14 |
| 9.4433 | 7.39 |
| 11.0719 | 11.34 |
| 11.9144 | 73.29 |
| 12.3921 | 29.17 |
| 12.5717 | 22.93 |
| 12.8791 | 8.93 |
| 13.6790 | 26.58 |
| 13.8694 | 15.67 |
| 14.4793 | 38.19 |
| 14.7398 | 55.26 |
| 14.9599 | 56.99 |
| 15.8715 | 20.66 |
| 16.4606 | 22.37 |
| 17.0459 | 20.43 |
| 17.4421 | 34.59 |
| 17.8203 | 46.78 |
| 18.2871 | 30.73 |
| 18.9573 | 43.91 |
| 19.4485 | 41.00 |
| 20.1190 | 35.53 |
| 20.7356 | 18.05 |
| 21.0535 | 30.09 |
| 21.6801 | 100.00 |
| 22.0236 | 18.06 |
| 22.7925 | 29.92 |
| 23.5044 | 41.92 |
| 23.9959 | 43.96 |
| 24.5555 | 31.47 |
| 25.1401 | 25.01 |
| 25.7588 | 59.24 |
| 26.0910 | 53.05 |
| 26.6137 | 39.47 |
| 27.5409 | 24.89 |
| 28.5493 | 22.44 |
| 29.1699 | 13.97 |
| 30.1441 | 21.01 |
| 31.2560 | 14.66 |
| 31.8783 | 16.47 |
| 32.7054 | 17.11 |
| 33.2797 | 24.40 |
| 33.9762 | 15.63 |

### Pharmacology

It has been found that the compound of the present invention blocks the interaction of menin with MLL proteins and oncogenic MLL fusion proteins. Therefore the compound according to the present invention and the pharmaceutical compositions comprising such compound may be useful for the treatment or prevention, in particular treatment, of diseases such as cancer, including but not limited to leukemia, myelodysplastic syndrome (MDS), and myeloproliferative neoplasms (MPN); and diabetes.

In particular, the compound according to the present invention and the pharmaceutical compositions thereof may be useful in the treatment or prevention of cancer. According to one embodiment, cancers that may benefit from a treatment with menin/MLL inhibitors of the invention comprise leukemias, lymphomas, myelomas or solid tumor cancers (e.g. prostate cancer, lung cancer, breast cancer, pancreatic cancer, colon cancer, liver cancer, melanoma and glioblastoma, etc.). In some embodiments, the leukemias include acute leukemias, chronic leukemias, myeloid leukemias, myelogenous leukemias, lymphoblastic leukemias, lymphocytic leukemias, Acute myelogenous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, leukemias exhibiting HOX/MEIS1 gene expression signatures etc.

In particular, the compound according to the present invention and the pharmaceutical compositions thereof may be useful in the treatment or prevention of myelodysplastic syndrome (MDS) or myeloproliferative neoplasms (MPN).

In particular, the compound according to the present invention and the pharmaceutical compositions thereof may be useful in the treatment or prevention of leukemias, in particular nucleophosmin (NPM1)-mutated leukemias, e.g. NPM1c.

In particular, the compound according to the present invention and the pharmaceutical compositions thereof may be useful in the treatment or prevention of AML, in particular nucleophosmin (NPM1)-mutated AML (i.e., NPM1^{mut} AML), more in particular abstract NPM1-mutated AML.

In particular, the compound according to the present invention and the pharmaceutical compositions thereof may be useful in the treatment or prevention of MLL-rearranged leukemias, in particular MLL-rearranged AML or ALL.

In particular, the compound according to the present invention and the pharmaceutical compositions thereof may be useful in the treatment or prevention of leukemias with MLL gene alterations, in particular AML or ALL with MLL gene alterations.

In particular, the compound according to the present invention and the pharmaceutical compositions thereof may be suitable for Q.D. dosing (once daily).

In particular, the compound according to the present invention and the pharmaceutical compositions thereof may be useful in the treatment or prevention of hematological cancer in a subject exhibiting NPM1 gene mutations and/or mixed lineage leukemia gene (MLL; MLL1; KMT2A) alterations, mixed lineage leukemia (MLL), MLL-related leukemia, MLL-associated leukemia, MLL-positive leukemia, MLL-induced leukemia, rearranged mixed lineage leukemia, leukemia associated with a MLL, rearrangement/alteration or a rearrangement/alteration of the MLL gene, acute leukemia, chronic leukemia, myelodysplastic syndrome (MDS), myeloproliferative neoplasms (MPN), insulin resistance, pre-diabetes, diabetes, or risk of diabetes, hyperglycemia, chromosomal rearrangement on chromosome 11q23, type-1 diabetes, type-2 diabetes; promoting proliferation of a pancreatic cell, where pancreatic cell is an islet cell, beta cell, the beta cell proliferation is evidenced by an increase in beta cell production or insulin production; and for inhibiting a menin-MLL interaction, where the MLL fusion protein target gene is HOX or MEIS1 in human.

Hence, the invention relates to the compound of the present invention for use as a medicament.

The invention also relates to the use of the compound of the present invention, for the manufacture of a medicament.

The present invention also relates to the compound according to the present invention, or a pharmaceutical composition according to the invention, for use in the treatment, prevention, amelioration, control or reduction of the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

Also, the present invention relates to the use of the compound according to the present invention, for the manufacture of a medicament for treating, preventing, ameliorating, controlling or reducing the risk of disorders associated with the interaction of menin with MLL proteins and oncogenic MLL fusion proteins in a mammal, including a human, the treatment or prevention of which is affected or facilitated by blocking the interaction of menin with MLL proteins and oncogenic MLL fusion proteins.

The invention also relates the compound according to the present invention, for use in the treatment or prevention of any one of the diseases mentioned hereinbefore.

The invention also relates to the compound according to the present invention, for use in treating or preventing any one of the diseases mentioned hereinbefore.

The invention also relates to the use of the compound according to the present invention, for the manufacture of a medicament for the treatment or prevention of any one of the disease conditions mentioned hereinbefore.

The compound of the present invention can be administered to mammals, preferably humans, for the treatment or prevention of any one of the diseases mentioned hereinbefore.

In view of the utility of the compound according to the present invention, there is provided a method of treating warm-blooded animals, including humans, suffering from any one of the diseases mentioned hereinbefore.

Said method comprises the administration, i.e. the systemic or topical administration, of a therapeutically effective amount of the compound according to the present invention, to warm-blooded animals, including humans.

Therefore, the invention also relates to a method for the treatment or prevention of any one of the diseases mentioned hereinbefore comprising administering a therapeutically effective amount of compound according to the invention to a patient in need thereof.

One skilled in the art will recognize that a therapeutically effective amount of the compound of the present invention is the amount sufficient to have therapeutic activity and that this amount varies inter alias, depending on the type of disease, the concentration of the compound in the therapeutic formulation, and the condition of the patient. An effective therapeutic daily amount would be from about 0.005 mg/kg to 100 mg/kg. The amount of a compound according to the present invention, also referred to herein as the active ingredient, which is required to achieve a therapeutically effect may vary on case-by-case basis, for example with the particular compound, the route of administration, the age and condition of the recipient, and the particular disorder or disease being treated. A method of treatment may also include administering the active ingredient on a regimen of between one and four intakes per day. In these methods of treatment the compound according to the invention is preferably formulated prior to administration.

The present invention also provides compositions for preventing or treating the disorders referred to herein. Said compositions comprising a therapeutically effective amount of a the compound according to the present invention, and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions may be prepared by any methods well known in the art of pharmacy, for example, using methods such as those described in Gennaro et al. Remington's Pharmaceutical Sciences (18^{th} ed., Mack Publishing Company, 1990, see especially Part 8 Pharmaceutical preparations and their Manufacture).

The compounds of the present invention may be administered alone or in combination with one or more additional therapeutic agents. Combination therapy includes administration of a single pharmaceutical dosage formulation which contains a compound according to the present invention and one or more additional therapeutic agents, as well as administration of the compound according to the present invention and each additional therapeutic agent in its own separate pharmaceutical dosage formulation.

Therefore, an embodiment of the present invention relates to a product containing as first active ingredient a compound according to the invention and as further active ingredient one or more anticancer agent, as a combined preparation for simultaneous, separate or sequential use in the treatment of patients suffering from cancer.

The one or more other medicinal agents and the compound according to the present invention may be administered simultaneously (e.g. in separate or unitary compositions) or sequentially in either order. In the latter case, the two or more compounds will be administered within a period and in an amount and manner that is sufficient to ensure that an advantageous or synergistic effect is achieved. It will be appreciated that the preferred method and order of administration and the respective dosage amounts and regimes for each component of the combination will depend on the particular other medicinal agent and compound of the present invention being administered, their route of administration, the particular condition, in particular tumour, being treated and the particular host being treated.

### Pharmacological Studies

In the pharmacological studies described below the following Compounds are described:
Compound A: (R)—N-ethyl-5-fluoro-N-isopropyl-2-((5-(2-(6-((2-methoxyethyl)(methyl)-amino)-2-methylhexan-3-yl)-2,6-diazaspiro[3.4]octan-6-yl)-1,2,4-triazin-6-yl)oxy)-benzamide;
Compound A1: (R)—N-ethyl-5-fluoro-N-isopropyl-2-((5-(2-(6-((2-methoxyethyl)(methyl)-amino)-2-methylhexan-3-yl)-2,6-diazaspiro[3.4]octan-6-yl)-1,2,4-triazin-6-yl)oxy)-benzanilide 0.2 HCl·x H₂O (x=2-3);
Compound A3: (R)—N-ethyl-5-fluoro-N-isopropyl-2-((5-(2-(6-((2-methoxyethyl)(methyl)-amino)-2-methylhexan-3-yl)-2,6-diazaspiro[3.4]octan-6-yl)-1,2,4-triazin-6-yl)oxy)-benzamide oxalate salt.
Compound A4: Crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate

The results from these pharmacological studies clearly show the biological activity of (R)—N-ethyl-5-fluoro-N-isopropyl-2-((5-(2-(6-((2-methoxyethyl)(methyl)-amino)-2-methylhexan-3-yl)-2,6-diazaspiro[3.4]octan-6-yl)-1,2,4-triazin-6-yl)oxy)-benzamide.

### 1) Menin/MLL Homogenous Time-Resolved Fluorescence (HTRF) Assay

To an untreated, white 384-well microtiter plate was added 40 nL 200× test compound in DMSO and 4 μL 2× terbium chelate-labeled menin (vide infra for preparation) in assay buffer (40 mM Tris-HCl, pH 7.5, 50 mM NaCl, 1 mM DTT (dithiothreitol) and 0.05% Pluronic F-127). After incubation of test compound and terbium chelate-labeled menin for 30 min at ambient temperature, 4 μL 2×FITC-MBM1 peptide (FITC-β-alanine-SARWRFPARPGT-NH₂) (SEQ ID NO: 3) (“FITC” means fluorescein isothiocyanate) in assay buffer was added, the microtiter plate centrifuged at 1000 rpm for 1 min and the assay mixtures incubated for 15 min at ambient temperature. The relative amount of menin-FITC-MBM1 complex present in an assay mixture is determined by measuring the homogenous time-resolved fluorescence (HTRF) of the terbium/FITC donor/acceptor fluorophore pair using an EnVision microplate reader (ex. 337 nm/terbium em. 490 nm/FITC em. 520 nm) at ambient temperature. The degree of fluorescence resonance energy transfer (the HTRF value) is expressed as the ratio of the fluorescence emission intensities of the FITC and terbium fluorophores (F^{em} 520 nm/F^{em} 490 nm). The final concentrations of reagents in the binding assay are 200 pM terbium chelate-labeled menin, 75 nM FITC-MBM1 peptide and 0.5% DMSO in assay buffer. Dose-response titrations of test compounds are conducted using an 11 point, four-fold serial dilution scheme, starting typically at 10 μM.

Compound potencies were determined by first calculating % inhibition at each compound concentration according to equation 1:

$$\% inhibition = ( ( HC - LC ) - ( {HTRF}^{compound} - LC ) ) / ( HC - LC ) ) * 100 \qquad ( Eqn 1 )$$

where LC and HC are the HTRF values of the assay in the presence or absence of a saturating concentration of a compound that competes with FITC-MBM1 for binding to menin, and HTRF^{compound} is the measured HTRF value in the presence of the test compound. HC and LC HTRF values represent an average of at least 10 replicates per plate. For each test compound, % inhibition values were plotted vs. the logarithm of the test compound concentration, and the IC₅₀ value derived from fitting these data to equation 2:

$$\% inhibition = Bottom + ( Top - Bottom ) / ( 1 + 10 \hat{} ( ( log {IC}_{50} - log [ cmpd ] ) * h ) ) \qquad ( Eqn 2 )$$

where Bottom and Top are the lower and upper asymptotes of the dose-response curve, respectively, IC₅₀ is the concentration of compound that yields 50% inhibition of signal and h is the Hill coefficient.

Preparation of Terbium cryptate labeling of Menin: Menin (a.a 1-610-6×his tag (SEQ ID NO: 2), 2.3 mg/mL in 20 mM Hepes (2-[4-(2-Hydroxyethyl)-1-piperazinyl]ethane sulfonic acid), 80 mM NaCl, 5 mM DTT (Dithiothreitol), pH 7.5) was labeled with terbium cryptate as follows. 200 μg of Menin was buffer exchanged into 1× Hepes buffer. 6.67 μM Menin was incubated with 8-fold molar excess NHS (N-hydroxysuccinimide)-terbium cryptate for 40 minutes at room temperature. Half of the labeled protein was purified away from free label by running the reaction over a NAP5 column with elution buffer (0.1M Hepes, pH 7+0.1% BSA (bovine serum albumin)). The other half was eluted with 0.1M phosphate buffered saline (PBS), pH7. 400 μl of eluent was collected for each, aliquoted and frozen at −80° C. The final concentration of terbium-labeled Menin protein was 115 μg/mL in Hepes buffer and 85 μg/mL in PBS buffer, respectively.

MENIN Protein Sequence (SEQ ID NO: 1):
MGLKAAQKTLFPLRSIDDVVRLFAAELGREEPDLVLLSLVLGFVEHFLA
VNRVIPTNVPELTFQPSPAPDPPGGLTYFPVADLSIIAALYARFTAQIR
GAVDLSLYPREGGVSSRELVKKVSDVIWNSLSRSYFKDRAHIQSLFSFI
TGTKLDSSGVAFAVVGACQALGLRDVHLALSEDHAWVVFGPNGEQTAEV
TWHGKGNEDRRGQTVNAGVAERSWLYLKGSYMRCDRKMEVAFMVCAINP
SIDLHTDSLELLQLQQKLLWLLYDLGHLERYPMALGNLADLEELEPTPG
RPDPLTLYHKGIASAKTYYRDEHIYPYMYLAGYHCRNRNVREALQAWAD
TATVIQDYNYCREDEEIYKEFFEVANDVIPNLLKEAASLLEAGEERPGE
QSQGTQSQGSALQDPECFAHLLRFYDGICKWEEGSPTPVLHVGWATFLV
QSLGRFEGQVRQKVRIVSREAEAAEAEEPWGEEAREGRRRGPRRESKPE
EPPPPKKPALDKGLGTGQGAVSGPPRKPPGTVAGTARGPEGGSTAQVPA
PAASPPPEGPVLTFQSEKMKGMKELLVATKINSSAIKLQLTAQSQVQMK
KQKVSTPSDYTLSFLKRQRKGLHHHHHH

### 2a) Proliferation Assay

The anti-proliferative effect of menin/MLL protein/protein interaction inhibitor test compounds was assessed in human leukemia cell lines. The cell line MOLM-14 harbors a MLL translocation and expresses the MLL fusion protein MLL-AF9, respectively, as well as the wildtype protein from the second allele. OCI-AML3 cells that carry the NPM1c gene mutation were also tested. MLL rearranged cell lines (e.g. MOLM-14) and NPM1c mutated cell lines exhibit stem cell-like HOXA/MEIS1 gene expression signatures. KO-52 was used as a control cell line containing two MLL (KMT2A) wildtype alleles in order to exclude compounds that display general cytotoxic effects.

MOLM-14 cells were cultured in RPMI-1640 (Sigma Aldrich) supplemented with 10% heat-inactivated fetal bovine serum (HyClone), 2 mM L-glutamine (Sigma Aldrich) and 50 μg/ml gentamycin (Gibco). KO-52 and OCI-AML3 cell lines were propagated in alpha-MEM (Sigma Aldrich) supplemented with 20% heat-inactivated fetal bovine serum (HyClone), 2 mM L-glutamine (Sigma Aldrich) and 50 μg/ml gentamycin (Gibco). Cells were kept at 0.3-2.5 million cells per ml during culturing and passage numbers did not exceed 20.

In order to assess the anti-proliferative effects, 200 MOLM-14 cells, 200 OCI-AML3 cells or 300 KO-52 cells were seeded in 200 μl media per well in 96-well round bottom, ultra-low attachment plates (Costar, catalogue number 7007). Cell seeding numbers were chosen based on growth curves to ensure linear growth throughout the experiment. Test compounds were added at different concentrations and the DMSO content was normalized to 0.3%. Cells were incubated for 8 days at 37° C. and 5% CO₂. Spheroid like growth was measured in real-time by live-cell imaging (IncuCyteZOOM, Essenbio, 4× objective) acquiring images at day 8. Confluence (%) as a measure of spheroid size was determined using an integrated analysis tool.

In order to determine the effect of the test compounds over time, the confluence in each well as a measure of spheroid size, was calculated. Confluence of the highest dose of a reference compound was used as baseline for the LC (Low control) and the confluence of DMSO treated cells was used as 0% cytotoxicity (High Control, HC).

Absolute IC₅₀ values were calculated as percent change in confluence as follows:
LC=Low Control: cells treated with e.g. 1 μM of the cytotoxic agent staurosporin, or e.g. cells treated with a high concentration of an alternative reference compound;
HC=High Control: Mean confluence (%) (DMSO treated cells);

$$\% Effect = 100 - ( 100 * ( Sample - LC ) / ( HC - LC ) ) ;$$

and
GraphPad Prism (version 7.00) was used to calculate the IC₅₀. Dose-response equation was used for the plot of % Effect vs Log 10 compound concentration with a variable slope and fixing the maximum to 100% and the minimum to 0%.
   2b) MEIS1 mRNA Expression Assay

MEIS1 mRNA expression upon treatment of compound was examined by Quantigene Singleplex assay (Thermo Fisher Scientific). This technology allows for direct quantification of mRNA targets using probes hybridizing to defined target sequences of interest and the signal is detected using a Multimode plate reader Envision (PerkinElmer). The MOLM-14 cell line was used for this experiment. Cells were plated in 96-well plates at 3,750 cells/well in the presence of increasing concentrations of compounds. After incubation of 48 hours with compounds, cells were lysed in lysis buffer and incubated for 45 minutes at 55° C. Cell lysates were mixed with human MEIS1 specific capture probe or human RPL28 (Ribosomal Protein L28) specific probe as a normalization control, as well as blocking probes. Cell lysates were then transferred to the custom assay hybridization plate (Thermo Fisher Scientific) and incubated for 18 to 22 hours at 55° C. Subsequently, plates were washed to remove unbound materials followed by sequential addition of preamplifiers, amplifiers, and label probe. Signals (=gene counts) were measured with a Multimode plate reader Envision. IC₅₀s were calculated by dose-response modelling using appropriate software. For all non-housekeeper genes response equal counts corrected for background and relative expression. For each sample, each test gene signal (background subtracted) was divided by the normalization gene signal (RPL28: background subtracted). Fold changes were calculated by dividing the normalized values for the treated samples by the normalized values for the DMSO treated sample. Fold changes of each target gene were used for the calculation of IC₅₀s.

The results are summarized below in Table 5.

TABLE 5
Biological data - HTRF, Proliferation and MEIS1 mRNA Expression Assays

| | HTRF- | | spheroid | OCI- | spheroid |
|---|---|---|---|---|---|
| | 30 min | MEIS1 | assay_OneTime | AML3 | assay_OneTime |
| Compound | incubation | IC₅₀ | MOLM-14 IC₅₀ | IC₅₀ | KO-52 IC₅₀ |
| Number | IC₅₀ (nM) | (μM) | (μM) | (μM) | (μM) |
| A | 0.09 | 0.02 | 0.021 | 0.091 | 6.85 |
| A3 | 0.098 | 0.017 | 0.017 | 0.12 | 7.75 |
| A1 | 0.18 | 0.017 | 0.011 | 0.08 | |
| A4 | | 0.011 | 0.008 | 0.019 | 5.44 |

### 3) Mouse PK (In Vivo T_{1/2} and Oral Bioavailability)

In vivo pharmacokinetics (PK) were assessed in fasted male CD-1 mice (age 6-8 weeks) following a single intravenous (IV, 0.5 or 1.0 mg/kg administered at 2.5 ml/kg) or oral (PO, 5 mg/kg administered at 10 ml solution/kg) dose of test article formulated in a 20% (w:vol) HP-β-CD solution or in Pyrogen free water.

Plasma and/or whole blood samples were collected from the dorsal metatarsal vein at desired timepoints via serial capillary microsampling (approx. 0.03 mL) using EDTA as an anticoagulant. Concentrations of compound in the plasma and blood samples were analyzed using a qualified LC-MS/MS method. In silico analysis of main pharmacokinetic parameters was performed using WinNonlin (Phoenix™, version 6.1) or similar software.)

### 4) Metabolic Stability in Human/Mouse Liver Microsomes

### Experimental Procedure

The objective of this study is to measure in vitro metabolic stability of test compound(s) in human and mouse liver microsomes and provide quantitative information on the rate of metabolic turnover (i.e. determination of the apparent intrinsic clearance of test).

Test items were prepared at a stock concentration of 10 mM in DMSO. For determination of metabolic turnover, a final working solution was prepared by adding 2 μL of 10 mM DMSO stock solution for test compound or positive control compounds to 198 μL of acetonitrile (100 μM final concentration).

Incubations were performed as follows: First, liver microsomes were thawed on ice and a master solution containing liver microsomes in 100 mM PBS (phosphate-buffered saline) at pH 7.4 is prepared. Next, the liver microsomes solution was added to the incubation plates and 10 mM NADPH (Nicotinamide-adenine dinucleotide phosphate) was added (MW: 833.4 g/mol; Roche Diagnostics GmbH, Germany. Dissolved in phosphate buffer (100 mmol/L, pH 7.4)). The mixture was mixed for 10 seconds and pre-warmed in the incubation plate at 37° C. for 10 minutes. The metabolic reaction was initiated with the addition of 5 μL of the 100 μM working solution for test compound or positive control compounds to incubation plate (final test item concentration=1 μM). The reaction final mixture should contain 1 mM NADPH, 0.5 mg/mL microsomes protein and 1 μM test compound or positive control compound in 100 mM PBS at pH 7.4. The percentage of organic solvent in incubation mixture is 1% with DMSO 0.02%. The reaction was quenched by transferring 50 μL of the incubated mixture at selected time points into the quenching plate containing 200 μL of cold methanol. After sampling of all the timepoints the quenching plate was centrifuged at 4000 rpm for 40 minutes to precipitate protein. A total of 90 μL of the supernatant was transferred to an analysis plate and ultra-pure H₂O water is added into each well for LC/MS/MS analysis. All incubations and analysis were performed in duplicate.

### Data Analysis

All calculations were carried out using Microsoft Excel. The slope value, k, was determined by linear regression of the natural logarithm of the remaining percentage of the parent drug vs. incubation time curve. The results are summarized below in Table 6.

The in vitro half-life (in vitro t_{1/2}) was determined from the slope value:

$$in vitro {t}_{1 / 2} = - ( 0.693 / k )$$

Conversion of the in vitro t_{1/2} (in min) into the in vitro intrinsic clearance (in vitro CLᵢₙₜ, in μL/min/mg proteins) was done using the following equation:

$$in vitro {Cl}_{int} = ( \frac{0.693}{{t}_{\frac{1}{2}}} ) * ( \frac{volume of incubation ( μL )}{amount of proteins ( mg )} )$$

TABLE 6
Mouse PK and Metabolic Stability

| | | In vivo | Bio- | | |
|---|---|---|---|---|---|
| | Formu- | T_{1/2} | avail- | Human | Mouse |
| Example | lating | (IV) | ability | LM Clint | LM Clint |
| number | agent | (h) | (PO) (%) | (μl/min/mg) | (μl/min/mg) |
| A | HP-β-CD | 6.7 | 17 | 19 | <7.5 |
| A3 | Pyrogen | 9.0 | 34 | 19 | <7.5 |
| | free water | | | | |
| 11 | HP-β-CD | NA | NA | 14 | <7.5 |

“NA” means not analyzed

### 5) Protocol for Pharmacodynamics (PD) Activity in Subcutaneous (Sc or SC) Xenografts of MOLM-14 or OCI-AML3 Cells

### Test Agents and Controls

Compound A3 was formulated in 20% hydroxypropyl-beta-cyclodextrin (HP-β-CD) and prepared to reach a total volume of 0.2 mL (10 mL/kg) per dose for a 20 g animal. Doses were adjusted by individual body weight each day. Working stocks of Compound A3 were prepared once per week for each study and stored at room temperature. Compound A3 was administered orally (PO), daily.

### Assay

The in vivo pharmacodynamics (PD) activity of compounds was evaluated in subcutaneous (SC) xenografts of MOLM-14 cells or OCI-AML3 cells. Nude NMRI mice (Crl:NMRI-Foxn1nu/−) harboring MOLM-14 or OCI-AML3 tumors were treated with 3 daily doses of vehicle or compounds. Plasma samples were collected at 23 hours after day 2 dose, 0.5 hours post final dose, and 16 hours post final dose and tumor samples were collected 16 hours post final dose. To examine the effects of compounds on the expression of multiple Menin-MLL target genes (e.g. MEIS1, MEF2C, FLT3) QuantiGene Plex technology (Thermo Fisher Scientific) was used. Frozen tumors were homogenized and transferred to individual lysing matrix tubes in lysis buffer and incubated for 30 minutes at 55° C. Cell lysates were mixed with target-specific capture probes, Luminex beads, and blocking probes, transferred to the custom assay hybridization plate (Thermo Fisher Scientific) and incubated for 18 to 22 hours at 54° C. Subsequently, plates were transferred to a magnetic separation plate and washed to remove unbound materials from beads followed by sequential hybridization of preamplifiers, amplifiers, and label probe and subsequent streptavidin phycoerythrin binding. Signals from the beads were measured with a Luminex FlexMap three-dimensional instrument. For all non-housekeeper genes response equal counts corrected for background and relative expression. For each sample, each test gene signal (background subtracted) was divided by the normalization gene signal (RPL19, RPL28, ATP6V1A: background subtracted). Fold changes were calculated by dividing the normalized values for the treated samples by the normalized values for the DMSO treated sample. The results are summarized below in Tables 7 and 8.

TABLE 7
Expression Level (% relative to vehicle) of Selected Genes
from MOLM-14 SC Model (mean values and standard deviations)

| Compound A3 | | | |
|---|---|---|---|
| (mg/kg) | MEIS1 | FLT3 | MEF2C |
| 0 | 101.30 ± 15.06 | 104.80 ± 10.07 | 103.50 ± 11.02 |
| 3 | 83.49 ± 25.48 | 78.67 ± 20.74 | 85.50 ± 22.77 |
| 10 | 62.84 ± 4.06 | 74.91 ± 8.97 | 68.04 ± 14.43 |
| 30 | 23.16 ± 2.75 | 52.61 ± 4.51 | 27.83 ± 2.17 |
| 50 | 14.40 ± 3.39 | 36.14 ± 3.50 | 18.75 ± 2.38 |
| 100 | 10.97 ± 3.21 | 35.82 ± 1.10 | 14.18 ± 1.56 |

TABLE 8
Expression Level (% relative to vehicle) of Selected Genes from
OCI-AML3 SC Model (mean values and standard deviations).

| Compound A3 (mg/kg) | MEIS1 |
|---|---|
| 0 | 100.30 ± 8.53 |
| 3 | 87.90 ± 39.75 |
| 10 | 48.81 ± 15.30 |
| 30 | 32.66 ± 3.71 |
| 50 | 23.83 ± 1.34 |
| 100 | 16.76 ± 1.92 |

Tables 7a and 8a show median values based on repeated experiments in optimized conditions with fresh tumor samples.

TABLE 7a
Expression level (% relative to vehicle) of selected genes from
MOLM-14 SC model (Median values and Standard Deviations).

| Compound A3 (mg/kg) | MEIS1 | FLT3 | MEF2C |
|---|---|---|---|
| 0 | 100.0 ± 13.5 | 100.0 ± 10.1 | 100.0 ± 11.0 |
| 3 | 83.7 ± 22.8 | 89.2 ± 20.7 | 87.7 ± 22.8 |
| 10 | 49.3 ± 5.9 | 79.8 ± 9.0 | 64.6 ± 14.4 |
| 30 | 14.7 ± 3.9 | 54.5 ± 4.5 | 28.8 ± 2.2 |
| 50 | 4.7 ± 1.1 | 37.6 ± 3.5 | 18.8 ± 2.4 |
| 100 | 3.3 ± 1.4 | 35.4 ± 1.1 | 13.6 ± 1.6 |

TABLE 8a
Expression level (% relative to vehicle) of selected gene from
OCI-AML3 SC model (Median values and Standard Deviations).

| Compound A3 (mg/kg) | MEIS1 |
|---|---|
| 0 | 100.0 ± 11.2 |
| 3 | 71.2 ± 15.1 |
| 10 | 26.5 ± 4.3 |
| 30 | 25.1 ± 11.2 |
| 50 | 8.5 ± 2.2 |
| 100 | 9.4 ± 1.2 |

### 6) Efficacy Study in MOLM-14 Subcutaneous Model

### Test Agents and Controls

Compound A3 was formulated in 20% hydroxypropyl-beta-cyclodextrin (HP-3-CD) and prepared to reach a total volume of 0.2 mL (10 mL/kg) per dose for a 20 g animal. Doses were adjusted by individual body weight each day. Working stocks of Compound A3 were prepared once per week for each study and stored at 25° C.

### Animals

Female NMRI Nude mice (MOLM-14 SC) were used when they were approximately 6 to 8 weeks of age and weighed approximately 25 g. All animals could acclimate and recover from any shipping-related stress for a minimum of 7 days prior to experimental use. Autoclaved water and irradiated food were provided ad libitum, and the animals were maintained on a 12 hour light and dark cycle. Cages, bedding, and water bottles were autoclaved before use and changed weekly. Further details are provided below in Table 9.

TABLE 9
Tissue Culture and Cell Injection Reagents

DPBS (Dulbecco's phosphate-buffered saline)
Heat-inactivated fetal bovine serum
RPMI 1640 medium
L-glutamine
Gentamycin
T175 Culture Flask
Roller Bottle

### Tumor Model and Cell Culture Method

Human AML cells MOLM-14 were cultured at 37° C., 5% CO₂ in the indicated complete culture media (RPMI 1640+10% HI-FBS+2 mM L-glutamine+50 ug/ml Gentamycin). Cells were harvested while in logarithmic growth and resuspended in cold (4° C.) Roswell Park Memorial Institute (RPMI) 1640 in serum-free medium.

Each mouse received 5×10⁶ MOLM-14 cells in 50% Matrigel in the right flank, in a total volume of 0.2 mL using a 1 cc syringe and a 27-gauge needle.

### Study Designs

Compound A3 was administered orally (PO), daily.

Day 0 is the day of tumor cell implantation and study initiation.

Mice bearing SC MOLM-14 tumors were randomized on Day 16 post-tumor implantation and assigned to treatment groups according to tumor volume (mean of ˜130 mm³; n=10/group). Treatment with vehicle or Compound A3 (at 30 and 100 mg/kg) was initiated on the same day, with daily oral dosing for 21 days. Plasma was collected at 1, 2, 4, 8, and 23 hours after the last dose (n=4-5/group/time point) for PK (pharmacokinetics) analysis.

### Animal Monitoring

SC tumor volume were measured for each animal 2 to 3 times per week or more throughout the study.

### Calculations

Tumor volume was calculated using the formula:

Tumor volume (mm³)=(D×d²/2); where ‘D’ represents the larger diameter and ‘d’ the smaller diameter of the tumor as determined by caliper measurements. Tumor volume data was graphed as the mean tumor volume±SEM.

The % ΔTGI was defined as the difference between mean tumor burden of the treatment and control groups, calculated as % ΔTGI=([(TV_{c}TVc₀)(TVₜTVₜ₀)]/(TV_{c}TVc₀))×100 where ‘TV_{c}’ is the mean tumor burden of a given control group, ‘TVc₀’ is the mean initial tumor burden of a given control group, ‘TVₜ’ is the mean tumor burden of the treatment group, and ‘TVₜ₀’ is the mean initial tumor burden of the treatment group. % TGI was defined as the difference between.

Mean tumor volumes of the treated and control groups, calculated as:

% TGI=((TV_{c}TVₜ)/TV_{c})×100 where ‘TV_{c}’ is the mean tumor volume of the control group and ‘TVₜ’ is the mean tumor volume of the treatment group. As defined by National Cancer Institute criteria, ≥60% TGI is considered biologically significant.

The % Tumor Regression (TR), quantified to reflect the treatment-related reduction of tumor volume as compared to baseline independent of the control group, was calculated as % TR=(1−mean (TVₜi/TVₜ₀i))×100 where ‘TVₜi’ is the tumor burden of individual animals in a treatment group, and ‘TVₜ₀i’ is the initial tumor burden of the animal.

### Data Analysis

Tumor volume were graphed using Prism software (GraphPad version 7 or 8). Statistical significance for most studies was evaluated for Compound A3-treated groups compared with HPβCD vehicle-treated controls on the last day of the study when ⅔ or more mice remained in each group. Differences between groups were considered significant when p≤0.05.

Statistical significance for animal tumor volume was calculated using the linear mixed-effects (LME) analysis in R software version 3.4.2 (using Janssen's internally developed Shiny application version 4.0), with treatment and time as fixed effects and animal as random effect.

Logarithmic transformation was performed if individual longitudinal response trajectories were not linear.

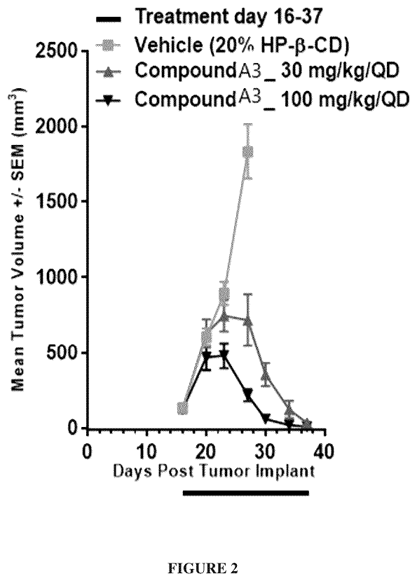

The information derived from this model was used to make pairwise treatment comparisons of tumor volumes to that of the control group or between all the treatment groups. The results are shown in FIG. 2.

7) Cardio-Electrophysiological Effects of the Testing Compounds in Synchronously Beating Human Pluripotent Stem Cell-Derived Cardiomyocytes (hSC-CMs) Using a Ca²⁺-Fluorescence Assay (CTCM human)

### Protocol

Compounds were tested in the 96-well plates.

Compounds were tested at 0.1 μM, 0.2 μM, 0.5 μM, 1 μM, 2.5 μM and 5 μM (n=4 per dose) on Cor.4U®-Cardiomyocytes or on iCell® Cardiomyocytes2.

Alternatively, compounds were tested at 0.1 μM, 0.3 μM; 1 μM, 3 μM, 10 μM and 30 μM (n=4 per dose) mostly on iCell® Cardiomyocytes2.

### Positive and Negative Controls

| | |
|---|---|
| Dofetilide | at 3 nM |
| Isoproterenol | at 100 nM |
| Nimodipine | at 100-300 nM |
| Cetirizine | at 3 μM. |

Vehicle control: Dimethylsulfoxide (DMSO). The solutions of the compound in DMSO or its solvent (final concentration of 0.1% DMSO; n=8).

### Preparation of Test Article and Controls

Tested compounds were dissolved in DMSO at 1000-fold the intended concentrations. A compound “mother-plate” was made, containing the test compounds and positive and negative controls at 1000-fold the final concentrations. At the experiment day, these stock solutions were diluted with Tyrode (Sigma), supplemented with 10 mM HEPES (Gibco), to 2-fold the intended concentration (in round bottom compound plates). Final DMSO concentration in test solutions and vehicle control was 0.1%.

### Cells

hSC-CMs (Cor.4U® Cardiomyocytes) were obtained from CDI (Ncardia, Germany). Cells are pre-plated and seeded in fibronectin-coated 96-well plates at a density suited to form a monolayer and maintained in culture in a stage incubator (37° C., 5% CO₂), according to the instructions of the cell provider.

Second line hSC derived cardiomyocyte called iCell® Cardiomyocytes2 were purchased from FUJIFILM Cellular Dynamics (USA). The experiments with test drugs are carried out 5 to 7 days after plating the cells onto the plate to have a living, beating monolayer of hiPSC-derived cardiomyocytes. The beating monolayer in 96-well-plates are normally taken from 2 Vials of frozen iCell® Cardiomyocytes2 (≈5 million cells/vial), which will be plated onto three 96-well plates (≈50K/well).

### Before Start of Experiment

At least one hour before the start of the experiments the normal cell medium was replaced with Tyrode solution with Calcium dye (see below).

Cal 520 dye (AAT Bioquest) was dissolved in 11 ml of Tyrode supplemented with 10 mM HEPES and warmed up to 37 C before adding to the cells.

35 μl cell culture medium was removed from each well and replaced with 35 μl of pre-warmed Cal 520 dye solution and cell plate was incubated for 45 min at 37° C./5% CO₂. Cells were incubated for 5 min at 37° C.

### Experiment

Spontaneous electrical activity is recorded, using Cal520™ (AAT Bioquest) calcium fluorescence-dye signaling. This dye integrates the total intracellular calcium activity over the whole well. A bottle of Cal520 dye (50 μg, MW: 1103/mol) is dissolved with 50 μl DMSO as a stock solution of 0.9 mM. 50 μL of the stock solution of the dye was added to 10 ml Tryodes solution to have dye concentration of 4.5 μM. Subsequently, 35 μl of this dye solution was added into each well, to have a final dye concentration of 1.58 μM. The current dye protocol on this CTCM human assay was established recently (Ivan Kopljar et al, Journal of Pharmacological and toxicological methods 2018. 91: 80-86; Lu et al., Tox Sci 2019. 170 (2): 345-356).

Fluorescent signals (Ca²⁺ transient morphology) were measured using the Functional Drug Screen System (FDSS/pCell; Hamamatsu, Japan) and the recordings were subsequently analyzed off-line, using appropriate software e.g. Notocord.

The cell plate was loaded into the FDSS/pCell for a test run: Ca²⁺ transients were measured for 4 minutes to check for synchronous beating of the cardiomyocytes in each well. All 96 wells were measured simultaneously (sampling interval: 0.06 s, short exposure time: 10 ms; excitation wavelength 480 nm; emission wavelength 540 nm; FDSS/pCell warmed to 37° C.). When all showed synchronous beating, the 96-well plate was measured repeatedly for 3 times (to verify synchronous beating in all 96-well at baseline, wells that did not meet the preset criteria were excluded from the study and not treated with compound):
T=0: control period (−5 to −1 min)+compound addition, followed for 3 min.
T=30: measured from 29 to 34 min after compound addition

During the compound addition step, 100 μl of the respective double-concentrated test solutions was pipetted into each well simultaneously.

Data were analyzed off-line using appropriate software e.g. Notocord-Hem (version 4.3).

The following parameters of the Ca²⁺ transient morphology were measured:
beat rate (BR)
amplitude of the Ca²⁺ transient (Amp),
CTD₉₀: Ca²⁺ transient duration at 90% (time to 90% of the initial base value).

The presence of various ‘arrhythmia-like’ activities were also noted during the experimental periods. These included:
‘early afterdepolarization-like’ (EAD-like) events (defined as “an extra small peak of the transient waveform following the initial peak of the transient”),
‘ventricular tachycardia-like’ (VT-like) events (defined as a very fast beating rate) or
‘ventricular fibrillation-like’ (VF-like) events (defined as “small amplitude, fast-rate Ca²⁺ waveforms with irregularities and non-measurable transient potentials)
‘cessation of beating’ of the cells (no Ca²⁺ transients observed).

If compound-induced changes on the calcium transient signal could not be analyzed by the software, then these signals were identified as BQL (below quality analyses level).

### Data Analysis

Data, measured from the FDSS-pCell, were copied for off-line analysis and were analyzed and uploaded in SPEC-II (our operational management system) for further analysis. The values of the variables before and after administration of the compound were collected and transferred into an Excel workbook.

All values (actual units and percentage changes from the baseline values) are expressed as median (minimum and maximum). Changes versus the corresponding baseline values (in actual units) observed in the compound group were compared with those in the solvent control group using the Wilcoxon-Mann-Whitney Test. Two-tailed tests with Bonferroni correction for multiplicity adjustment were conducted. Since there are 10 treatment groups each compared to the solvent group, alpha level of 0.05/10 (0.005) was considered to reflect a statistically significant difference from the solvent group. All statistical analysis was performed using appropriate software e.g. R software version 3.5.2.

Quality Control of the hiPSC-CMs in the plate:

Plates were rejected if they did not meet following criteria:
Stable regular beating
Amplitude >500 relative units
Beat rate between 25 and 80 beats per minute
CTD₉₀ between 300 and 800 ms.

In the present study, the hiPSC-CMs in the plates met the above criteria.

These parameters combined with incidence of arrhythmia or cessation of beating were used to calculate the potential hazard level using a weighted scoring method (based on Kopljar et al., Stem Cell Reports 2018. 11, 1365-1377). This hazard score is calculated per concentration by adding weighted points based on the Tolerance Intervals (TI) on the changes of CTD₉₀, the beat rate and amplitude (ΔΔ %) and incidence of beating stop and early afterdepolarization (EAD). Consequently, for each concentration one of four different hazard levels will be generated. This will be done after 30-min of incubated with compound. The hazard levels are:

No hazard: within the vehicle effect levels or small non-relevant changes.

Low hazard: relevant effect but potentially low risk for cardiac liabilities.

High hazard: relative high risk for cardiac liabilities.

Very high hazard: very high risk due to arrhythmic like events (EAD's).

The ‘Hazard Score’ results provide an identification for potential acute cardiac drug-induced effects at free drug equivalent (as no plasma proteins are added to the wells). Evaluation of hazard identification is conducted using a ‘scoring reference book’ called CTCM_Scoring_version 1 (Kopljar et al., Stem Cell Reports 2018. 11: 1365-1377), and levels are indicated according to the following color scheme of Table 10.

TABLE 10
Color Schemes of Hazardous Identification Legend

| | |
|---|---|
| Green | No concern |
| Yellow | Low concern |
| Red | High concern |
| Black | Very high concern due to arrhythmic events |

Ranking of a testing compound according to hazard score severity on the Ca²⁺ transient assay measured in HiPSc-CMs as listed above in different colors and in the associated table.

### Results

Using iCell® Cardiomyocytes2 as Cell Line

Positive and negative controls: The positive and negative controls all had expected pharmacological effects in this assay. The results are summarized below in Tables 11 and 12.

TABLE 11
Hazard Scoring for Compound A3

| | Color @ | Color @ | Color @ | Color @ | Color @ | Color @ |
|---|---|---|---|---|---|---|
| Compound | 0.1 μM | 0.2 μM | 0.5 μM | 1 μM | 2.5 μM | 5 μM |
| A3 | Green | Green | Green | Green | Green | Green |

TABLE 12
Hazard Scoring for Compound A1

| | Color @ | Color @ | Color @ | Color @ | Color @ | Color @ |
|---|---|---|---|---|---|---|
| Compound | 0.1 μM | 0.3 μM | 1 μM | 3 μM | 10 μM | 30 μM |
| A1 | Green | Green | Green | Green | Green | yellow |

For compound A1: with an efficacious dose in mouse xenograft models of 30 mpk (mg/kg), CTCM human concentration vs free Cₘₐₓ would be estimated as follows:
Margin CTCM human 10 μM vs free Cmax>16 (mouse, human)
Margin CTCM human 30 μM vs free Cmax>45 (mouse, human).
   8) Effect on the Membrane Potassium Current I_{Kr} in hERG Transfected Cell Lines Protocol 1:

### Abbreviations

CHO Chinese hamster ovary cell line
DMSO Dimethylsulfoxide
hERG human ether-à-go-go-related gene
I_{Kr} rapidly activating delayed-rectifier K⁺ current

### Methods

Experiments were performed using CHO cells stably expressing the hERG potassium channel. Cells were grown at 37° C. and 5% CO₂ in culture flasks in Ham's F12 Medium supplemented with 10% heat-inactivated fetal calf serum, hygromycin B (100 μg/ml) and geneticin (100 μg/ml). For use in the automated patch-clamp system QPatch (Sophion) cells were harvested to obtain cell suspension of single cells.

Solutions: The bath solution contained (in mM) 145 NaCl, 4 KCl, 10 glucose, 10 HEPES ((4-(2-hydroxyethyl)-1-piperazineethanesulfonic acid), 2 CaCl₂ and 1 MgCl₂ (pH 7.4 with NaOH).

The pipette solution contained (in mM) 120 KCl, 10 EGTA (Ethylene glycol-bis(2-aminoethylether)-N,N,N′,N′-tetraacetic acid), 10 HEPES, 5.374 CaCl₂ and 1.75 MgCl₂ (pH 7.2 with KOH).

Patch-clamp experiments were performed in the voltage-clamp mode and whole-cell currents were recorded with an automated patch-clamp assay utilizing the QPatch system (Sophion).

Current signals were amplified and digitized, stored and analyzed by using the QPatch assay software.

The holding potential was −80 mV. The hERG current (K⁺-selective outward current) was determined as the maximal tail current at −40 mV after a 2 second depolarization to +60 mV. Pulse cycling rate was 15 s. A short pulse (90 ms) to −40 mV served as a baseline step to calculate the tail current amplitude. After establishing whole-cell configuration and a stability period, the solvent control (0.3% DMSO) was applied for 5 minutes followed by the test substance by four increasing concentrations of 3×10⁻⁷ M, 3×10⁻⁶ M, 10⁻⁵ M and 3×10⁻⁵ M. Each concentration of the test substance was applied twice. The effect of each concentration was determined after 5 min as an average current of 3 sequential voltage pulses. To determine the extent of block the residual current was compared with vehicle pre-treatment. Concentration/response relations were calculated by non-linear least-squares fits to the individual data points. The half-maximal inhibiting concentration (IC50) was calculated by the fitting routine.

Each compound was replicated on the same plate in at least 5 wells. Percent inhibition of at The results are summarized below in Table 13.

TABLE 13
hERG IC₅₀ (μM) from Protocol 1

| Compound Number | hERG- IC₅₀ (μM) |
|---|---|
| A | >30.2 |

### 9) Efficacy Study in Disseminated OCI-AML3 Model

### Test Agents and Controls

Compound A3 was formulated in 20% hydroxypropyl-beta-cyclodextrin (HP-3-CD) and prepared to reach a total volume of 0.2 mL (10 mL/kg) per dose for a 20 g animal. Doses were adjusted by individual body weight each day. Working stocks of Compound A3 were prepared once per week for each study and stored at 25° C.

### Animals

Female SCID beige mice (CB17.Cg-PrkdcscidLystbg-J/Crl/−) were used when they were approximately 6 to 8 weeks of age and weighed approximately 25 g. All animals could acclimate and recover from any shipping-related stress for a minimum of 7 days prior to experimental use. Autoclaved water and irradiated food were provided ad libitum, and the animals were maintained on a 12 hour light and dark cycle. Cages, bedding, and water bottles were autoclaved before use and changed weekly. The tissue culture and cell injection reagents are summarized below in Table 14.

TABLE 14
Tissue Culture and Cell Injection Reagents

DPBS (Dulbecco's phosphate-buffered saline)
Heat-inactivated fetal bovine serum
MEM Alpha medium
L-glutamine
Gentamycin
T175 Culture Flask
Roller Bottle

### Tumor Model and Cell Culture Method

Human AML cell line OCI-AML3 was cultured at 37° C., 5% CO₂ in the indicated complete culture media (MEM Alpha+20% HI—FBS (Heat-Inactivated Fetal Bovine Serum)+2 mM L-glutamine+50 ug/ml Gentamycin). Cells were harvested while in logarithmic growth and resuspended in cold (4° C.) MEM ((Minimum Essential Medium) Alpha in serum-free medium. For the disseminated OCI-AML3 model, each mouse received 5×10⁵ cells via IV injection in a total volume of 0.2 mL using a 26-gauge needle.

### Study Designs

Compound A3 was administered orally (PO), daily.

Day 0 is the day of tumor cell implantation and study initiation.

In the efficacy study, mice bearing IV OCI-AML3 xenograft tumors were randomly assigned to treatment groups 3 days post-tumor cell engraftment. Treatment with vehicle or Compound A3 (at 30, 50,100 mg/kg) was initiated on the same day, with daily dosing for 28 days.

### Animal Monitoring

Animals were monitored daily for clinical signs related to either compound toxicity or tumor burden (i.e., hind limb paralysis, lethargy, etc.).

### Calculations

For survival assessment, results were plotted as the percentage survival against days post tumor implant. Negative clinical signs and/or ≥20% body weight loss was used as a surrogate endpoint for death. Median survival was determined utilizing Kaplan-Meier survival analysis. The percent increased life span (ILS) was calculated as: ((median survival day of treated group−median survival day of control group)/median survival day of control group)×100. Animals failing to reach the surrogate endpoint due to adverse clinical signs (such as ulcerated tumors, body weight loss, etc.) or death unrelated to treatment were censored for the survival assessment. As defined by NCI criteria, ≥25% ILS is considered biologically significant. (Johnson J I et al. Br J Cancer. 2001. 84(10), 1424-1431).

### Data Analysis

Survival and body weight data were graphically represented utilizing Prism (Version 7).

Statistical significance for body weights was evaluated as described above. Statistical significance was evaluated for Kaplan-Meier survival plots comparing therapeutic treatment group vs. appropriate vehicle-treated control using log-rank (Mantel-Cox) test in R software version 3.4.2. Differences between groups were considered significant when the p value was ≤0.05.

### Survival

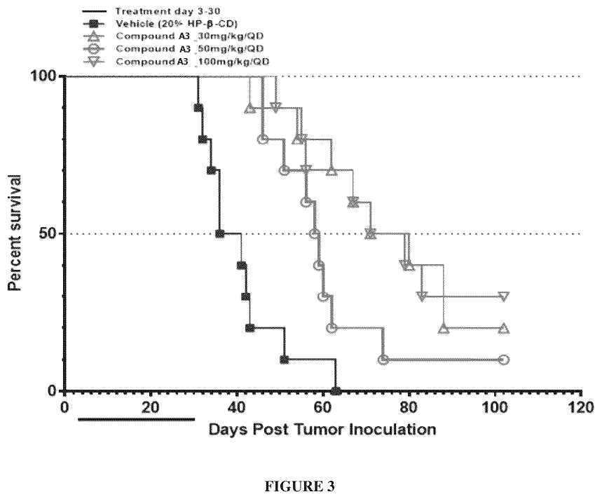

The Kaplan-Meier survival curve is shown in in FIG. 3. Mice bearing established OCI-AML3 tumors were orally dosed daily with Compound A3 at 30, 50, 100 mg/kg in 20% HP-β-CD formulation for a total of 28 days (n=9-10/group). For Compound A3 treated groups, the median days of survival were reached at the following days for 30 mg/kg at day 75.5, for 50 mg/kg at day 58.5 and for 100 mg/kg at day 75 this compared to a median survival of 38.5 days for the vehicle-treated control group. Compound A3 treatment resulted in statistically significant increased lifespan of OCI-AML3 tumor-bearing mice by 96.1%, 51.9% and 94.8% (at the 30, 50 and 100 mg/kg dose levels) as compared to that of control mice, (p≤0.001). This was a biologically significant ILS as per NCI criteria threshold of ≥25% ILS (Johnson J I et al. Br J Cancer. 2001. 84(10), 1424-1431).

### Stability Data

Stability experiments were performed for crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate. The bis-besylate salt hydrate is found to be chemically and physically stable with no degradation observed by UHPLC and no solid-state change observed by XRD under evaluated stress conditions.

| | Besylate salt Purity | XRD |
|---|---|---|
| Reference | 99.65 | — |
| 50° C./10% RH, 7 days | 99.64 | — |
| 50° C./10% RH, 14 days | 99.62 | — |
| 50° C./50% RH, 7 days | 99.65 | — |
| 50° C./50% RH, 14 days | 99.66 | — |
| Reference | 99.21 | Crystalline, form A, ref |
| 50° C./30% RH, 21 days | 99.32 | Complies to ref |
| 50° C./75% RH, 21 days | 99.36 | Complies to ref |

1. (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide besylate salt 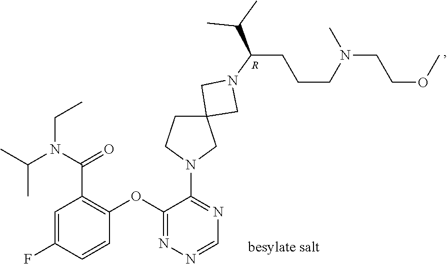
or a solvate thereof.

2. The compound according to claim 1 wherein the solvate is a hydrate.

3. The compound according to claim 1 wherein the compound is a crystalline form A of (R)—N-ethyl-5-fluoro-N-isopropyl-2-((5-(2-(6-((2-methoxyethyl)(methyl)amino)-2-methylhexan-3-yl)-2,6-diazaspiro[3.4]octan-6-yl)-1,2,4-triazin-6-yl)oxy)benzamide bis-besylate salt hydrate,
wherein the crystalline form produces an X-ray powder diffraction pattern comprising peaks at 5.4, 7.2, 11.1, 11.9, and 21.7 degrees two theta±0.2 degrees two theta.

4. The crystalline form of claim 3, wherein the X-ray powder diffraction pattern may further comprise at least one peak selected from 13.7, 14.5, 14.7, 15.0, 16.5, 17.8, 19.0, 19.4, and 20.1 degrees two theta±0.2 degrees two theta.

5. The crystalline form of claim 3, further characterized by an X-ray powder diffraction pattern substantially as depicted in FIG. 1.

6. A pharmaceutical composition comprising a compound of claim 1 and at least one of a pharmaceutically acceptable carrier, a pharmaceutically acceptable excipient, and a pharmaceutically acceptable diluent.

7. A process for preparing a pharmaceutical composition as defined in claim 6 comprising mixing a pharmaceutically acceptable carrier with a therapeutically effective amount of a compound according to claim 1.

8. A compound as claimed in claim 1 use as a medicament.

9. A compound as claimed in claim 1 for use in the prevention or treatment of cancer.

10. The compound as claimed in claim 9 where the cancer is leukemia, myelodysplastic syndrome (MDS), and myeloproliferative neoplasms (MPN).

11. The compound for use according to claim 10 in the prevention or treatment of leukemia wherein the leukemia is (NPM1)-mutated leukemia.

12. The compound for use according to claim 9, wherein cancer is selected from leukemias, lymphomas, myelomas or solid tumor cancers such as prostate cancer, lung cancer, breast cancer, pancreatic cancer, colon cancer, liver cancer, melanoma and glioblastoma.

13. The compound for use according to claim 10, wherein the leukemia is selected from acute leukemias, chronic leukemias, myeloid leukemias, myelogenous leukemias, lymphoblastic leukemias, lymphocytic leukemias, Acute myelogenous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX/MEIS1 gene expression signatures.

14. A method of treating or preventing cancer, comprising administering to a subject in need thereof, a therapeutically effective amount of a compound as claimed in claim 1.

15. A process for preparing the crystalline form of claim 3, comprising the step of recrystallising (R)—N-ethyl-5-fluoro-N-isopropyl-2-((5-(2-(6-((2-methoxyethyl) (methyl)amino)-2-methylhexan-3-yl)-2,6-diazaspiro[3.4]octan-6-yl)-1,2,4-triazin-6-yl)oxy) benzamide (Compound A) wherein the recrystallisation comprises the steps of:
a) adding Compound A, or a hydrate or solvate thereof, to a mixture of suitable solvents, in the presence of benzenesulfonic acid, and adjusting to a temperature in the range of from about 20° C. to solvent reflux temperature;
b) seeding with crystalline form A;
c) yielding a precipitate of the crystalline form of claim 3.

16. The process of claim 15, wherein the mixture of suitable solvents is a mixture of acetone, water and IPAc.

17. The process of claim 15, wherein the mixture of suitable solvents is a mixture of isopropanol, water and IPAc.

18. The process of claim 15 wherein the temperature is about 25° C.

19. A crystalline form of 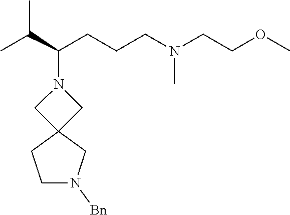 [SMILES: COCCN(C)CCC[C@H](C(C)C)N1CC2(CCN(Cc3ccccc3)C2)C1]
Bn citric acid salt,
wherein the crystalline form produces an X-ray powder diffraction pattern comprising peaks at 5.82, 10.09 and 18.42 degrees two theta±0.2 degrees two theta.

20. A method to provide N-ethyl-5-fluoro-2-hydroxy-N-isopropylbenzamide via a one step reaction by reacting 5-fluoro-2-hydroxy-benzoic acid in the presence of the coupling agent CDI, in a suitable solvent:

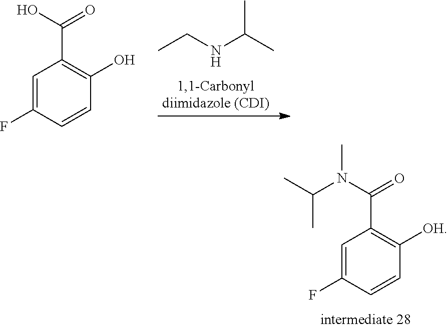

21. The crystalline form of claim 4, further characterized by an X-ray powder diffraction pattern substantially as depicted in FIG. 1.

22. The pharmaceutical composition as claimed in claim 6 for use as a medicament.

23. The pharmaceutical composition as claimed in claim 6 for use in the prevention or treatment of cancer.

24. The pharmaceutical composition as claimed in claim 23 where the cancer is leukemia, myelodysplastic syndrome (MDS), and myeloproliferative neoplasms (MPN).

25. The pharmaceutical composition as claimed in claim 24 for use according to claim 10 in the prevention or treatment of leukemia wherein the leukemia is (NPM1)-mutated leukemia.

26. The pharmaceutical composition for use according to claim 23, wherein the cancer is selected from leukemias, lymphomas, myelomas or solid tumor cancers such as prostate cancer, lung cancer, breast cancer, pancreatic cancer, colon cancer, liver cancer, melanoma and glioblastoma.

27. The pharmaceutical composition for use according to claim 24, wherein the leukemia is selected from acute leukemias, chronic leukemias, myeloid leukemias, myelogenous leukemias, lymphoblastic leukemias, lymphocytic leukemias, Acute myelogenous leukemias (AML), Chronic myelogenous leukemias (CML), Acute lymphoblastic leukemias (ALL), Chronic lymphocytic leukemias (CLL), T cell prolymphocytic leukemias (T-PLL), Large granular lymphocytic leukemia, Hairy cell leukemia (HCL), MLL-rearranged leukemias, MLL-PTD leukemias, MLL amplified leukemias, MLL-positive leukemias, and leukemias exhibiting HOX/MEIS1 gene expression signatures.

26. A method of treating or preventing cancer, comprising administering to a subject in need thereof, a therapeutically effective amount of a pharmaceutical composition as claimed in claim 6.


SEQUENCE LISTING (3 sequences)

SEQ ID NO: 1 - protein, 616 aa, Artificial Sequence
  Description of Artificial Sequence Synthetic polypeptide
MGLKAAQKTLFPLRSIDDVVRLFAAELGREEPDLVLLSLVLGFVEHFLAVNRVIPTNVPELTFQPSPAPDPPGGLTYFPVADLSIIAALYARFTAQIRGAVDLSLYPREGGVSSRELVKKVSDVIWNSLSRSYFKDRAHIQSLFSFITGTKLDSSGVAFAVVGACQALGLRDVHLALSEDHAWVVFGPNGEQTAEVTWHGKGNEDRRGQTVNAGVAERSWLYLKGSYMRCDRKMEVAFMVCAINPSIDLHTDSLELLQLQQKLLWLLYDLGHLERYPMALGNLADLEELEPTPGRPDPLTLYHKGIASAKTYYRDEHIYPYMYLAGYHCRNRNVREALQAWADTATVIQDYNYCREDEEIYKEFFEVANDVIPNLLKEAASLLEAGEERPGEQSQGTQSQGSALQDPECFAHLLRFYDGICKWEEGSPTPVLHVGWATFLVQSLGRFEGQVRQKVRIVSREAEAAEAEEPWGEEAREGRRRGPRRESKPEEPPPPKKPALDKGLGTGQGAVSGPPRKPPGTVAGTARGPEGGSTAQVPAPAASPPPEGPVLTFQSEKMKGMKELLVATKINSSAIKLQLTAQSQVQMKKQKVSTPSDYTLSFLKRQRKGLHHHHHH

SEQ ID NO: 2 - protein, 6 aa, Artificial Sequence
  Description of Artificial Sequence Synthetic peptide
HHHHHH

SEQ ID NO: 3 - protein, 13 aa, Artificial Sequence
  Description of Artificial Sequence Synthetic peptide
  MOD_RES (1)..(1): Beta-alanine
ASARWRFPARPGT